(19) United States
(12) Patent Application Publication
(10) Pub. No.: US 2023/0056427 A1
(43) Pub. Date: Feb. 23, 2023

(54) SYSTEMS AND METHODS FOR CUSTOMIZING ORTHODONTIC TREATMENT AND TREATMENT PLANNING

(71) Applicant: Align Technology, Inc., San Jose, CA (US)

(72) Inventors: Samuel BLANCO, Santa Clara, CA (US); Anton LAPSHIN, Nizhniy Novgorod (RU); Eric P. MEYER, Pleasanton, CA (US); Michael FLANAGAN, Redwood City, CA (US); Yury SLYNKO, Moscow (RU); Maxim SHALASHOV, Moscow (RU); Evgeniy MALASHKIN, Moscow (RU); Rowena CHEONG, Waterford Residence (SG); Roman A. ROSCHIN, Moscow (RU); Sergei LIUBIMOV, Cary, NC (US)

(21) Appl. No.: 17/885,356

(22) Filed: Aug. 10, 2022

Related U.S. Application Data
(60) Provisional application No. 63/231,703, filed on Aug. 10, 2021.
(60) Provisional application No. 63/340,436, filed on May 10, 2022.

(51) Int. Cl. A61C 7/00; G16H 20/40; G06F 3/0482; G06F 3/04847

(52) U.S. Cl. CPC A61C 7/002; G16H 20/40; G06F 3/0482; G06F 3/04847

(57) ABSTRACT

Systems and methods are disclosed for user modification of a rule or set of textual rules in plain language the reference instructions in a domain-specific treatment language that is used to generate dental appliance data. In some cases, the user may interact with a graphical user interface to modify a practitioner-specific subset of textural rules. The graphical user interface may allow modification of the practitioner-specific subset of textural rules without prior knowledge of the domain-specific treatment programming language. Each interaction region may cause sections of the predefined treatment preferences to be modified without knowledge of the dental protocol language. The example systems, methods, and/or computer-readable media described herein help with designing treatment plans for orthodontic treatments, including treatment plans based on predefined treatment preferences expressed in a dental protocol language.

### CLAIM OF PRIORITY

This patent application claims priority to U.S. Provisional Patent Application No. 63/231,703, titled “SYSTEMS AND METHODS FOR CUSTOMIZING TREATMENT PLANNING,” filed on Aug. 10, 2021, and U.S. Provisional Patent Application No. 63/340,436, titled “SYSTEMS AND METHODS FOR ORTHODONTIC TREATMENT,” filed on May 10, 2022, each of which is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

Treatment planning may be used in any medical procedure to help guide a desired outcome. For example, treatment planning may be used in orthodontic and dental treatments where a series of patient-removable appliances (e.g., orthodontic aligners, palatal expanders, and the like) are provided to correct a variety of different orthodontic or dental conditions, and in particular for treating malocclusions. Treatment planning is typically performed in conjunction with the dental professional (e.g., dentist, orthodontist, dental technician, etc.), by generating a model of the patient's teeth in a final configuration and then breaking the treatment plan into a number of intermediate stages (steps) corresponding to individual dental appliances (aligners) that are worn sequentially. This process may be interactive, adjusting the staging and in some cases the final target position, based on constraints on the movement of the teeth and the dental professional's preferences.

Existing systems and methods for treatment planning may be time consuming, and may provide only limited choices and control to the dental professional. The treatment plan may be determined, at least in part, by a treatment planning protocol. The treatment planning protocol may describe a clinician's preferred method or approach for orthodontic treatment. In some cases, the treatment planning protocol may be captured with a domain-specific treatment programming language that may be used to generate dental appliance data for the patient. The dental appliances may, in turn, be fabricated from the dental appliance data.

The domain-specific treatment programming language describing the treatment planning protocol, however, may not be easily understood by clinicians. Thus, modification of the domain-specific treatment programming language to match a clinician's personal preferences may be difficult.

### SUMMARY OF THE DISCLOSURE

The systems and methods described herein relate generally to the generation of dental appliance data, and more particularly to user modification of one or more treatment planning protocols used to generate the dental appliance data. For example, described herein are apparatuses (e.g., systems, devices, etc., including software and/or hardware, and non-transitory computer-readable storage medium comprising instructions) and methods for the generation of dental appliance data and more particularly for generating and treatment plans that are customized to a particular dental practitioner such as an orthodontist, dentist, dental technicians, or the like, or groups of any of these. In particular, described herein are methods and apparatuses that allow dental practitioners to easily and rapidly engage with dental- or orthodontic-treatment planning systems that may use a domain-specific treatment planning language to create treatment plans for aligning or otherwise modifying their patient's dentition. These methods and apparatuses may allow the user (e.g., the dental practitioner) to create and modify detailed and versatile sets of practitioner-specific textural rules that correlate to sets of instructions in a domain-specific treatment language without requiring that the user be trained in programming the domain-specific treatment language that may be used to control systems for generating treatment plans and/or forming aligners. Although the methods and apparatuses described herein may allow modification when forming practitioner-specific textural rules, these methods and apparatuses may guide the user(s) in generating these practitioner-specific textural rules in a manner that reduces the amount of iteration and modification necessary.

Also described herein are methods including methods of forming a set (or subset) of practitioner-specific textural rules. As used herein a practitioner may refer to the dental professional (e.g., dentist, orthodontist, etc.) and may refer to a single dental professional or a group of affiliated dental professionals (e.g., a dental practice or dental office).

In general, each practitioner-specific rule may include a plurality of treatment steps, which may modify a patient's teeth during a treatment plan. The steps of the treatment plan may be steps in a domain-specific treatment language (as described, for example, in U.S. patent application Ser. No. 16/178,491, titled “AUTOMATIC TREATMENT PLANNING,” filed Nov. 1, 2018 and herein incorporated by reference in its entirety). The rules may be conditional (e.g., if X condition is present in the patient's teeth, then apply particular steps), and/or specific to particular teeth or regions of the teeth, and/or particular stages in the treatment plan.

For example, a method may include: presenting a dental practitioner with a graphical user interface including a menu of textual rules specific to orthodontic treatment planning, wherein the rules refer to a collection of instructions in a domain-specific treatment language; receiving, from the dental practitioner, selections from the menu of textural rules to form a practitioner-specific subset of textural rules; applying the practitioner-specific subset of textural rules to a test dental data set to generate a test treatment plan; displaying the test treatment plan to the dental practitioner; modifying, based on input from the dental practitioner, the practitioner-specific subset of textural rules; and storing the practitioner-specific subset of textural rules to apply to one or more sets of patient data.

The menu of textual rules may include rules specific virtually any treatment, for example: tooth leveling, gap spacing, crowding, extraction, interproximal reduction, overjet, overbite, cross bite, attachments, and anterior-to-posterior correction.

Any of these methods and apparatuses may also include reconciling any conflict between the rules of the practitioner-specific subset of textural rules. The system may include a reconciling module. Reconciling may identify conflicting or contradictory rules and may either modify the rule to resolve the conflict and/or it may include apply or reference a hierarchy of rules and conditions to determine which rules to include (or in some cases present) to the dental practitioner.

In any of these methods and apparatuses, modifying may comprise receiving modifications to the test treatment plan and modifying the practitioner-specific textural rules accordingly. Alternatively or additionally, modifying may comprise receiving dental practitioner edits to the practitioner-specific subset of textural rules directly. The method or apparatus may iteratively modify the set (e.g., subset) of practitioner-specific textural rules.

Any of these methods and apparatuses may include generating a treatment plan specific to a patient by applying the practitioner-specific subset of textural rules to the patient's dental data. In some examples, the method may include fabricating dental appliances based on the treatment plan. For example, the method may include receiving patient data (e.g., scans, including intraoral scans, of the patient's dentition, generating one or more digital representation of the patient's dentition (including, but not limited to a 3D model of the patient's dentition, and/or segmented model(s) of the patient's dentition), and applying the practitioner-specific subset of textural rules to generate one (or more) treatment plans for the patient using the digital representation of the patient's teeth.

The method and apparatus may enhance the user (practitioner) experience by modifying information (e.g., the presented subset of a larger collection of textural rules) that are presented to the user to select from in the user interface. The larger collection of textural rules may be included in a data structure (e.g., data store, memory, etc.) that is remote or locally kept and accessed and may be updated and/or curated. The larger collection of textural rules may include rules generated by all or a large number of prior or current users (e.g., dentists, orthodontists, etc.) and/or by prior or current users that are considered experts. Within the larger set of textural rules there may be a number of overlapping and contradictory textural rules; in general the apparatus and methods described herein may present at least a subset of these rules by pre-determining for a particular user which of the larger collection of textural rules to present as options to a particular dental practitioner (user). Thus any of these methods and apparatuses may include selecting a subset of the textural rules for a specific dental practitioner based on one or more characteristic of the dental practitioner. Any appropriate characteristic may be used, including the size of the dental practitioner's practice, the number of dental aligner cases performed (historically and/or expected to be performed) by the dental practitioner, and a geographic location of the dental practitioner. This information may be provided by the dental practitioner as part of the processes described herein. In some cases the dental practitioner may indicate a preference for one or more know other dental practitioners and the apparatus or method may provide rules (and may so indicate for each rule) corresponding to a particular practitioner. In some cases the method or apparatus may select textural rules from the larger collection of rules that correlate with the country or region of a country; for example particular rules may be generated or commonly used by dental practitioners with these regions, and/or having a particular amount of experience, number of patients and/or practice size. Thus, in general, the larger collection of rules may include correlations (e.g., metadata) for each rule tracking the origin, use and/or region(s) with each rule.

As mentioned, in any of these apparatuses and method the subset of rules to be presented may be selected for the particular user. This process may also include removing rules that conflict. Alternatively conflicting rules may be presented, and the conflict may be resolved only if they are selected by the user, as mentioned.

In general, the method and apparatus may provide one or more graphical user interface to simplify and/or enhance the user's experience in selecting and/or modifying a practitioner-specific subset of textural rules. For example, the graphical user interface may include a user dialog box including a selectable list of the textual rules specific to orthodontic treatment planning.

The rules presented to the user may be presented as a menu of textual rules that are specific to orthodontic treatment planning, and that are shown as a curated list of textural rules organized by rank and/or by any of the metadata (e.g., correlation to one or more other users, including popularity, geographic preference, etc.). As mentioned, the rules may be taken from a curated rules database and selected based on one or more characteristic of the dental practitioner.

Any of these method or apparatuses may include applying the practitioner-specific subset of textural rules to the test dental data set to generate the test treatment plan comprises generating a plurality of dental aligners corresponding to each of a plurality of stages of the test treatment plan. For example, the method or apparatus may include providing one or more patient data set from the dental practitioner as the test dental data set. In some examples, the test dental data set may comprise a standardized dental data set (a “model” data set, for a particular scanned set of teeth).

In any of these methods and apparatuses displaying the test treatment plan to the dental practitioner may include displaying the menu of textural rules in a first window and displaying an image of teeth corresponding to the test dental data set at one or more stages of the test treatment plan in a second window so that the dental practitioner may modify the selection of textural rules from the menu of textural rules in the first window and may modify the view of the image of teeth corresponding to the test dental data set at one or more stages of the test treatment plan in the second window.

For example, a method may include: presenting a dental practitioner with a graphical user interface comprising a first window including a menu of textual rules specific to orthodontic treatment planning, wherein the rules refer to a collection of instructions in a domain-specific treatment language; receiving, from the dental practitioner, selections from the menu of textural rules to form a practitioner-specific subset of textural rules; reconciling any conflict between the rules of the practitioner-specific subset of textural rules; applying the practitioner-specific subset of textural rules to a test dental data set to generate a test treatment plan; displaying the test treatment plan to the dental practitioner in a second window, wherein the second window displays an image of teeth corresponding to the test dental data set at one or more stages of the test treatment plan; modifying, based on input from the dental practitioner, the practitioner-specific subset of textural rules; and generating a treatment plan specific to a patient by applying the practitioner-specific subset of textural rules to the patient's dental data.

Also described herein are apparatuses for performing any of these methods. For example described herein are apparatuses (e.g., devices or systems) comprising: one or more processors; and a memory configured to store instructions that, when executed by the one or more processors, cause the device to: present a dental practitioner with a graphical user interface including a menu of textual rules specific to orthodontic treatment planning, wherein the rules refer to a collection of instructions in a domain-specific treatment language; receive from the dental practitioner, selections from the menu of textural rules to form a practitioner-specific subset of textural rules; apply the practitioner-specific subset of textural rules to a test dental data set to generate a test treatment plan; display the test treatment plan to the dental practitioner; modify, based on input from the dental practitioner, to the practitioner-specific subset of textural rules; and store the practitioner-specific subset of textural rules to apply to one or more sets of patient data.

Also described herein is software (e.g., non-transitory computer-readable storage medium comprising instructions) for performing any of these methods. For example, described herein are non-transitory computer-readable storage media comprising instructions that, when executed by one or more processors of a device, cause the device to perform operations comprising: presenting a dental practitioner with a graphical user interface including a menu of textual rules specific to orthodontic treatment planning, wherein the rules refer to a collection of instructions in a domain-specific treatment language; receiving, from the dental practitioner, selections from the menu of textural rules to form a practitioner-specific subset of textural rules; applying the practitioner-specific subset of textural rules to a test dental data set to generate a test treatment plan; displaying the test treatment plan to the dental practitioner; modifying, based on input from the dental practitioner, the practitioner-specific subset of textural rules; and storing the practitioner-specific subset of textural rules to apply to one or more sets of patient data.

In general, these methods and apparatuses may also be used to generate the dental appliance data. The generation of the dental appliance data may be performed by a system or apparatus executing software instructions that may be guided by the treatment plan, which may be written in a domain-specific treatment programming language. The treatment plan may be generated for a specific set of dental data (e.g., patient data) based on the practitioner-specific subset of textural rules, as described above. A graphical user interface may be used to modify the treatment plan and/or the practitioner-specific subset of textural rules. In some cases, the graphical user interface may illustrate and describe rules and/or portions of the treatment planning protocol in a manner that is easily understood by a clinician. The graphical user interface may enable the clinician to change the treatment planning protocol without directly editing the domain-specific treatment programming language.

In one example, a method may include accessing a first patient's dental data, wherein the first patient's dental data includes a dental scan data associated with the first patient, accessing a set of rules (which may be referred to herein as a treatment planning protocol, or TPP) describing orthodontic treatment procedures, wherein the rules reference commands for treatment descriptions in an orthodontic treatment language, and generating dental appliance data for the first patient based, at least in part, on the first patient's dental data and the rule. The method may further include displaying predicted tooth positions associated with the dental appliance data for the first patient on a graphical user interface (GUI), modifying the rules based on a user interaction with the GUI, wherein the user interaction is in response to the displayed predicted tooth positions associated with the dental appliance data for the first patient; and regenerating dental appliance data for the first patient based on modified rules(s).

In some examples, the method may further include fabricating dental appliances based on the regenerated dental appliance data for the first patient. Furthermore, the fabricated dental appliances may include a plurality of clear dental aligners.

In some examples, displaying the predicted tooth positions may further include displaying a user dialog box associated with the rule(s) associated with the predicted displayed tooth positions. Furthermore, a user interaction with the user dialog box may modify at least one instruction of the orthodontic treatment language.

In some examples, the predicted tooth positions may be based, at least in part, on the rule(s) and the first patient's dental data. In some other examples, the method may further include saving the modified rule(s) as a custom rule for a user. In some examples, the accessed rule may be selected from the group consisting of a default rule, a rule associated with another user, and a user's custom rule. In some other examples, the dental appliance data may include data associated with a plurality of stages of orthodontic treatment. In still other examples, the accessed rule may be based, at least in part, on a user's questionnaire responses.

The method may further include displaying predicted tooth positions associated with the regenerated dental appliance data for the first patient and saving the practitioner-specific subset of textural rules and the regenerated dental appliance data. In some examples, the dental appliance data for the first patient may include data associated with a plurality of dental aligners.

In some examples, the method may include receiving a second patient's dental data, generating a dental appliance data for the second patient based, at least in part, on the second patient's dental data and the practitioner-specific subset of textural rules, and displaying predicted tooth positions associated with the dental appliance data for the second patient on the GUI. Furthermore, the method may include modifying the practitioner-specific subset of textural rules based on a user interaction with the GUI, wherein the user interaction is in response to the displayed predicted tooth positions associated with the dental appliance data for the second patient and regenerating dental appliance data for the second patient based on the modified practitioner-specific subset of textural rules.

In another example, a device may include one or more processors, and a memory configured to store instructions that, when executed by the one or more processors, cause the device to: access a first patient's dental data, wherein the first patient's dental data includes a dental scan data associated with the first patient, access a treatment planning protocol (e.g., rule) describing orthodontic treatment procedures, wherein the practitioner-specific subset of textural rules references instructions in an orthodontic treatment language, and generate dental appliance data for the first patient based, at least in part, on the first patient's dental data and the practitioner-specific subset of textural rules. The instructions, when executed may further cause the device to display predicted tooth positions associated with the dental appliance data for the first patient on a graphical user interface (GUI), modify the practitioner-specific subset of textural rules based on a user interaction with the GUI, wherein the user interaction is in response to the displayed predicted tooth positions associated with the dental appliance data for the first patient, and regenerate dental appliance data for the first patient based on modified rule.

In some examples, execution of the instructions may cause the device to further fabricate dental appliances based on the regenerated dental appliance data for the first patient. Furthermore, the fabricated dental appliances may include a plurality of clear dental aligners.

In some examples, execution of the instructions to display the predicted tooth positions may cause the device to further display a user dialog box associated with the rule associated with the predicted displayed tooth positions. Furthermore, a user interaction with the user dialog box may modify at least one instruction of the orthodontic treatment language.

In some examples, the predicted tooth positions may be based, at least in part, on the practitioner-specific subset of textural rules and the first patient's dental data. Furthermore, execution of the instructions may cause the device to save the modified practitioner-specific subset of textural rules as a custom practitioner-specific subset of textural rules for a user. In some examples, the accessed practitioner-specific subset of textural rules may be selected from the group consisting of a default rule, a rule associated with another user, and a user's custom rule. In some other examples, the dental appliance data may include data associated with a plurality of stages of orthodontic treatment. In some examples, the accessed rule may be based, at least in part, on a user's questionnaire responses.

In some other examples, execution of the instructions may cause the device to further display predicted tooth positions associated with the regenerated dental appliance data for the first patient and save the practitioner-specific subset of textural rules and the regenerated dental appliance data. In still other examples the dental appliance data for the first patient may include data associated with a plurality of dental aligners.

In some examples, execution of the instructions may cause the device to further receive a second patient's dental data, generate a dental appliance data for the second patient based, at least in part, on the second patient's dental data and the practitioner-specific subset of textural rules, and display predicted tooth positions associated with the dental appliance data for the second patient on the GUI. Furthermore, execution of the instructions may cause the device to further modify the practitioner-specific subset of textural rules based on a user interaction with the GUI, wherein the user interaction is in response to the displayed predicted tooth positions associated with the dental appliance data for the second patient and regenerate dental appliance data for the second patient based on the modified practitioner-specific subset of textural rules.

In some examples, a non-transitory computer-readable storage medium comprising instructions that, when executed by one or more processors of a device, may cause the device to perform operations comprising: accessing a first patient's dental data, wherein the first patient's dental data includes a dental scan data associated with the first patient, accessing a practitioner-specific subset of textural rules describing instructions for performing an orthodontic treatment procedures, wherein the practitioner-specific subset of textural rules references instructions for treatment descriptions in an orthodontic treatment language, and generating dental appliance data for the first patient based, at least in part, on the first patient's dental data and the practitioner-specific subset of textural rules. The device may further perform operations comprising displaying predicted tooth positions associated with the dental appliance data for the first patient on a graphical user interface (GUI), modifying the practitioner-specific subset of textural rules based on a user interaction with the GUI, wherein the user interaction is in response to the displayed predicted tooth positions associated with the dental appliance data for the first patient, and regenerating dental appliance data for the first patient based on modified practitioner-specific subset of textural rules.

In some examples, execution of the instructions may cause the device to perform operations further comprising fabricating dental appliances based on the regenerated dental appliance data for the first patient. Furthermore, the fabricated dental appliances may include a plurality of clear dental aligners.

In some examples, execution of the instructions may cause the device to perform operations further comprising displaying a user dialog box associated with the practitioner-specific subset of textural rules associated with the predicted displayed tooth positions. Furthermore, a user interaction with the user dialog box may modify at least one instruction of the orthodontic treatment language.

In some examples, the predicted tooth positions may be based, at least in part, on the practitioner-specific subset of textural rules and the first patient's dental data. In some other examples, execution of the instructions may cause the device to perform operations further comprising saving the modified practitioner-specific subset of textural rules as a custom practitioner-specific subset of textural rules for a user.

In some examples, the accessed practitioner-specific subset of textural rules may be selected from the group consisting of a default rule, a rule associated with another user, and a user's custom rule. In some other examples, the dental appliance data may include data associated with a plurality of stages of orthodontic treatment. In still other examples, the accessed rule may be based, at least in part, on a user's questionnaire responses.

In some examples, execution of the instructions may cause the device to perform operations further comprising displaying predicted tooth positions associated with the regenerated dental appliance data for the first patient and saving the practitioner-specific subset of textural rules and the regenerated dental appliance data. In some examples, the dental appliance data for the first patient may include data associated with a plurality of dental aligners.

In some examples, execution of the instructions may cause the device to perform operations further comprising receiving a second patient's dental data, generating a dental appliance data for the second patient based, at least in part, on the second patient's dental data and the practitioner-specific subset of textural rules, and displaying predicted tooth positions associated with the dental appliance data for the second patient on the GUI. Furthermore, execution of the instructions may cause the device to perform operations further comprising modifying the practitioner-specific subset of textural rules based on a user interaction with the GUI, wherein the user interaction is in response to the displayed predicted tooth positions associated with the dental appliance data for the second patient and regenerating dental appliance data for the second patient based on the modified practitioner-specific subset of textural rules.

As mentioned, in general, the methods and apparatuses described herein may relate to the technical field of treatment planning using domain-specific computer systems and methods, and more particularly to domain-specific computer systems and methods used for treatment planning, such as medical (e.g., dental, orthodontic, etc.) treatment planning.

The system, methods and/or computer-readable media described herein may be for planning a treatment for a patient. These treatment plans may include, but are not limited to, orthodontic treatment plans, such as treatment plan including one or more of: a shell aligner, a palatal expander, etc. These system, methods and/or computer-readable media described herein provide technical solutions to the highly technical problems of treatment planning, including medical treatment planning (e.g., dental treatment planning, orthodontic treatment planning, surgical treatment planning, orthotic treatment planning, etc.). Generally, these methods may include using a dental protocol language to encode user (e.g., physician, therapist, dentist, orthodontist, etc.) preferences as part of a treatment template (also referred to as a treatment protocol). A treatment planning engine may use the treatment planning instructions, along information about the patient (e.g., the patient's oral cavity, such as a scan of the patient's teeth or other relevant body regions) to automatically generate one or more treatment plans specific to the patient. Because the treatment plan(s) is/are generated using the treatment planning instructions derived from a user's customized treatment template, the resulting treatment plan(s) may also be customized to the user. The resulting treatment plans may be reviewed and approved by the user.

For example, the systems, methods, and/or computer-readable media described herein provide technical solutions to the highly technical problems of orthodontic treatment planning, and may include using a dental protocol language (e.g., a domain-specific orthodontic treatment language) to encode user (e.g., dentist, orthodontist, dental technician, etc.) preferences as part of a treatment template.

A treatment plan may refer to a series of steps, devices and/or schedules for altering a subject's physiology to achieve or approach a desired outcome. In some cases the treatment plan is an orthodontic treatment plan and may refer to a series of steps, devices and/or schedules for altering a subject's dental arch to achieve or approach a desired outcome. For convenience, in the examples described herein the orthodontic and/or dental treatment plans may be referred to as “orthodontic treatment plans,” or simply “treatment plans,” although it should be understood that other types of treatment plans may be included, such as surgical treatment plans, orthotic treatment plans, and the like.

Described herein are methods of generating an orthodontic treatment plan. Any of these methods may include: presenting a clinician with predefined treatment parameters for generating a treatment plan, wherein the predefined treatment parameters comprise a data structure in a dental protocol language, accepting, through a user interface, one or more modifications to the predefined treatment parameters, appending the one or more modifications to the data structure as a higher layer, transferring the data structure and a digital model of a patient's teeth to a treatment plan generating module, wherein the treatment plan generating module modifies the treatment parameters based on the higher layer and generates a modified set of treatment parameters, and displaying the treatment plan to the clinician for approval.

In any of the methods described herein, the modifications to the data structure may be encoded in the dental protocol language. In some examples, the methods described herein may include repeating the steps of presenting the clinician with the predefined treatment parameters and accepting one or more modifications following the display of the treatment plan to the clinician.

Any of the methods described herein may further include forming a series of aligners based on the approved treatment plan. Any of the methods described herein may include presenting the clinician with the predefined treatment preferences comprises generating or receiving the predefined treatment preferences from a library of clinician treatment preferences indexed by the clinician.

In some examples, the predefined treatment preferences of the methods described herein may be based on prior treatment plans approved by the clinician for other patients.

Also described herein is a non-transitory computer-readable storage medium comprising instructions that, when executed by one or more processors of a device, cause the device to perform operations comprising presenting a clinician with predefined treatment parameters for generating a treatment plan, wherein the predefined treatment parameters comprise a data structure in a dental protocol language, accepting, through a user interface, one or more modifications to the predefined treatment parameters, appending the one or more modifications to the data structure as a higher layer, transferring the data structure and a digital model of a patient's teeth to a treatment plan generating module, wherein the treatment plan generating module modifies the treatment parameters based on the higher layer and generates a modified set of treatment parameters, and displaying the treatment plan to the clinician for approval.

In any of the non-transitory computer-readable storage mediums described herein, the modifications to the predefined treatment parameters may be encoded in the dental protocol language.

In some examples, execution of the instructions may cause the device to perform operations further comprising repeating the steps of presenting the clinician with the predefined treatment parameters and accepting one or more modifications following the display of the treatment plan to the clinician.

In some examples, execution of the instructions may cause the device to perform operations further comprising forming a series of aligners based on the approved treatment plan. In some examples, presenting the clinician with the predefined treatment preferences may include generating or receiving the predefined treatment preferences from a library of clinician treatment preferences indexed by the clinician.

In some examples, in any of the non-transitory computer-readable storage mediums described herein, the predefined treatment preferences may be based on prior treatment plans approved by the clinician for other patients.

Also described herein are systems that may include one or more processors and a memory configured to store instructions that, when executed by the one or more processors, cause the system to present a clinician with predefined treatment parameters for generating a treatment plan, wherein the predefined treatment parameters comprise a data structure in a dental protocol language, accept, through a user interface, one or more modifications to the predefined treatment parameters, append the one or more modifications to the data structure as a higher layer, transfer the data structure and a digital model of a patient's teeth to a treatment plan generating module, wherein the treatment plan generating module modifies the treatment parameters based on the higher layer and generates a modified set of treatment parameters, and display the treatment plan to the clinician for approval.

In any of the systems described herein, the modifications to the data structure may be encoded in the dental protocol language. Furthermore, in any of the systems described herein the execution of the instructions may cause the system to repeat the steps of presenting the clinician with the predefined treatment parameters and accepting one or more modifications following the display of the treatment plan to the clinician.

In any of the systems described herein, the execution of the instructions causes the device to perform operations further comprising forming a series of aligners based on the approved treatment plan.

In any of the systems described herein, execution of the instructions to present the clinician with predefined treatment parameters for generating a treatment plan may cause the system to generate or receive the predefined treatment preferences from a library of clinician treatment preferences indexed by the clinician.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1 is a flowchart depicting an example method for generating dental appliance data, in accordance with some examples.

FIG. 2 shows a top view of clear dental aligners that may be fabricated from the dental appliance data described with respect to FIG. 1.

FIG. 3 is a flowchart depicting an example method for customizing a practitioner-specific subset of textural rules in accordance with some examples.

FIG. 4 shows an example of a rule, in accordance with some examples.

FIG. 5A shows a graphical user interface that may be used to help generate a practitioner-specific subset of textural rules.

FIG. 5B is another example of a user interface that may be used to help generate a practitioner-specific subset of textural rules.

FIG. 6 is a flowchart depicting an example method for generating dental appliance data, in accordance with some examples.

FIG. 7 shows an example graphical user interface including interactive treatment planning protocol modification selections, in accordance with some examples.

FIG. 8 shows a block diagram of a device that may be configured to perform the operations described herein.

FIG. 9 is a diagram showing an example of systems in a device planning environment.

FIG. 10 is a diagram showing an example of a system.

FIG. 11 shows an example user interface displaying selection choices for modifying treatment parameters.

FIG. 12 is diagram showing an example of elements of a domain-specific orthodontic treatment language.

FIGS. 13A-13B show an example of a user preference (in this example, posterior cross-bite) that may be written in a domain-specific orthodontic treatment language for a treatment template; in FIG. 13A the default preference is shown as improving posterior cross-bite, while FIG. 13B shows the preference for not improving posterior cross-bite.

FIGS. 14A-14B show an example of a user preference (in this example, bite ramp attachments) that may be written in a domain-specific orthodontic treatment language for a treatment template; in FIG. 14A the default preference is shown as no bite ramps, while FIG. 14B shows the preference for using attachments placed on premolars, with bite ramps for all cases except where there is an open bite and/or rotated laterals.

FIGS. 15A-15B show an example of a user preference (in this example, position of attachments on teeth) that may be written in a domain-specific orthodontic treatment language for a treatment template; in FIG. 15A the default preference is shown as locating attachments in a mid-range of the tooth, while FIG. 15B shows the preference for positioning the attachments close to the gingiva.

FIGS. 16A-16B show an example of a user preference (in this example, target overbite) that may be written in a domain-specific orthodontic treatment language for a treatment template; in FIG. 16A the default preference is shown as not correcting the target overbite, while FIG. 15B shows the preference for correcting the overbite to within a selected target (e.g., between 0.1 mm and 1 mm).

FIGS. 17A-17B show an example of a user preference (in this example, lingual bite ramp attachments) that may be written in a domain-specific orthodontic treatment language for a treatment template; in FIG. 17A the default preference is shown as not including lingual bite ramp attachments for anterior intrusion, while FIG. 17B shows the preference for including lingual bite ramp attachments for the lower anterior intrusion.

FIG. 18(i)-18(vi) is a table illustrating example grammar and diction for a domain-specific orthodontic treatment language.

FIG. 19 shows an example of a method for modifying a treatment plan.

FIG. 20 shows a block diagram of a device 1200 that may be an example of the system 1000 of FIG. 10.

### DETAILED DESCRIPTION

Apparatuses (e.g., systems, devices, etc.) and methods for generating dental appliance data are described herein. In some examples, the dental appliance data may be generated by an execution of one or more software modules that follow a clinician's practitioner-specific subset of textural rules to provide dental (orthodontic) treatment. As mentioned, the rules referred to here, which may also be referred to as textual rules, are written in text and each rule describes treatment steps (typically multiple treatment steps) in simple language that may be understood by the dental practitioner. Each rule refers to (and points to) a set of domain-specific language instructions that modify a patient's teeth during a treatment plan. Thus, a set of rules may define instructions for forming a treatment plan. The instructions referenced by the rules may be steps in a domain-specific treatment language (as described, for example, in U.S. patent application Ser. No. 16/178,491, titled “AUTOMATIC TREATMENT PLANNING,” filed Nov. 1, 2018 and herein incorporated by reference in its entirety). The rules, e.g., the practitioner-specific subset of textural rules, may be used with patient tooth date, such as a digital model of the patient's teeth, to generate a treatment plan, and this treatment plan may be used to generate a series of dental appliances (dental appliance data) to perform the treatment plan. The dental appliances may be fabricated as one or more aligners that may implement various stages of the clinician's treatment plan (treatment protocol).

The collection of rules specific to the practitioner, practitioner-specific subset of textural rules, may be collected and may encode a set of instructions written in a domain-specific treatment programming language to smoothly interface with one or more software modules to generate the treatment plan and/or series of aligners. As described herein, the user (clinician) may interact directly at the level of the rules to select and/or modify the practitioner-specific subset of textural rules. This is because modification of the set of instructions of the treatment planning protocol themselves may be difficult, particularly for clinicians unfamiliar with programming languages, much less the domain-specific treatment programming language of the treatment planning protocol. Described herein are various methods and apparatuses that enable a clinician to modify and customize a set of rules encoding instructions for a treatment planning protocol without requiring the clinician to be familiar with the domain-specific treatment programming language.

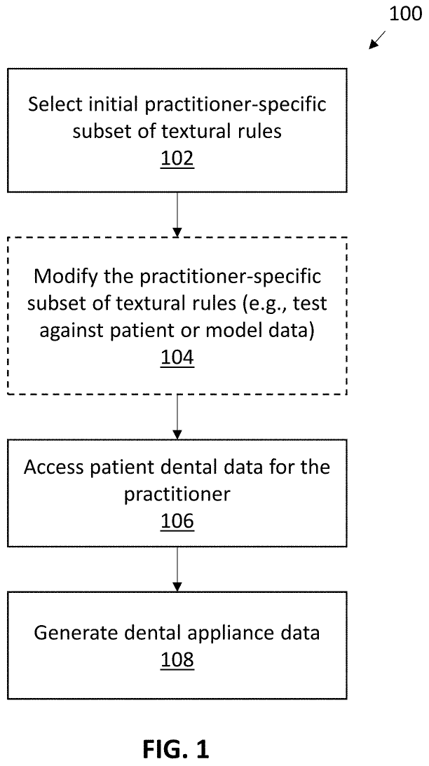

A general method for generating dental appliance data is described below in conjunction with FIG. 1. FIG. 1 is a flowchart depicting an example method 100 for generating dental appliance data, in accordance with some examples. Some examples may perform the operations described herein with additional operations, fewer operations, operations in a different order, operations in parallel, and some operations differently. The method 100 describes, in a general manner, the operations performed by, or at the direction of, a clinician seeking to generate dental appliance data that may be used to fabricate one or more dental appliances. The dental appliances may include, for example, one or more clear dental aligners that may be worn by a patient to deliver or provide any feasible orthodontic and/or dental treatment.

In some examples, the dental appliance data may be generated by a system comprising hardware, software or a combination of hardware and software that operates on patient dental data and is guided by a clinician's treatment protocol. The clinician's treatment protocol may describe the clinician's preferential steps and/or settings for providing the orthodontic and/or dental treatment.

The method in this example begins in block 102 where a clinician, also referred to as a dental practitioner (e.g., orthodontist, dentist, dental technician, or the like) selects an initial set of rules. A rule may describe the clinician's dental protocol for addressing the movement, rotation, and/or positioning of some of all of the teeth during all or a portion of a treatment. The rules may form a subset of rules that can implement the clinicians preferred orthodontic treatment technique. In some cases, the rule may include conditional statements to test for various conditions (detected spaces, tooth rotations, tooth inclinations, and the like) as well as dental treatments to be performed if various conditions are detected.

In some cases, the rule may be reference instructions in a domain-specific treatment (orthodontic) programming language that includes orthodontic specific constructs to describe orthodontic treatments (e.g., degrees of tooth rotation, bite ramp usage, deep bite correction, etc.). In some examples, the rule may be used to direct and/or control an apparatus, module, or system that generates dental appliance data. In some cases, the dental appliance data may be used to provide dental treatment for a patient. For example, the dental appliance data may enable the fabrication of one or more dental aligners. The dental aligners may be worn by a patient to affect tooth movement through a number of treatment stages.

The initial subset of practitioner-specific rules may be selected from any number of feasible sources. In some cases, the practitioner may form new rules. For example a practitioner may generate a rule based on an extensive initial (e.g., onboarding) interview between the clinician and a technician skilled in writing and describing dental protocols (instructions) in the domain-specific programming language used to describe the rule. For example, the technician may ask a battery of questions that may be used to determine some or all of the clinician's treatment preferences and express them as parameters and constructs of the domain-specific treatment programming language. In some cases, the interview may be lengthy due to the amount of the material that may be covered. In some other cases, the clinician may complete a questionnaire with questions corresponding to the battery of questions from the technician. As a result of the interview or the questionnaire, the technician may provide the clinician with the initial rule.

In some other cases, the clinician may select a default rule or set of rules as an initial rule or set of rules of the practitioner-specific subset of textural rules. The default rule or subset of rules may include default treatment settings for many or all of the parameters of the domain-specific treatment programming language. In some examples, one or more of the default rules may be regionally oriented and may be tailored for use by a dentist or an orthodontist.

In still other cases, the clinician may select a rule that may be provided by or based on another user's rule as the initial rule. For example, the clinician may want to follow a treatment protocol from a well-known user (e.g., an opinion leader). This user may have a great deal of experience in using the same or similar system used by the clinician. If the user is willing to share his or her rule, then other clinicians may copy and, if desired, modify the shared rule. Thus, a clinician can select the rule of another user as a basis for their own rule.

The rules described above may be stored locally with respect to the system described herein, or may be stored remotely, for example, through remote or cloud-based storage systems.

Next, in block 104, the clinician may modify their selected practitioner-specific subset of rules, either by adding or remove rules or by modifying a specific rule (including modifying the associated domain-specific treatment language instructions). Modification of the rule or practitioner-specific subset of textural rules may be optional as depicted here by a dashed line around block 104. In some cases, the modifications may be made with the help of a technician. For example, a technician, skilled in writing and describing dental protocols in the domain-specific treatment language, may interact with the clinician and modify the rule in accordance with any desired changes. In some other cases, the clinician may modify the rule directly.

In some cases the modification may be made by generating a sample treatment plan applying the practitioner-specific subset of textural rules to a digital dataset for a set of teeth (“model data”). The model data may be based on a known model, or may use an actual patient for the practitioner. The practitioner-specific subset of textural rules may be transmitted with the model data to a treatment plan generator that may translate the practitioner-specific subset of textural rules, into instructions and apply the instructions to the model data to generate a sample treatment plan. The practitioner may be presented with the treatment plan and may iteratively modify the practitioner-specific subset of textural rules (adding, removing and/or modifying specific rules) and re-run the treatment plan until satisfied with the resulting practitioner-specific subset of textural rules.

The practitioner-specific subset of textural rules may be saved for use against one or more actual patient data sets, and or immediately used, to generate treatment plans and associated dental appliances (e.g., aligners).

For example, in block 106, the clinician may access (retrieve) patient dental data. The patient data may include, for example, dental scan data, patient demographic data, or any other relevant patient data relating to the patient's dental or orthodontic physiology. Similar to the practitioner-specific subset of textural rules, the patient dental data may be stored locally or may be stored and retrieved from remote or cloud-based storage systems.

Next, in block 108, the clinician may generate dental appliance data. For example, the clinician may use a system (e.g., hardware, software, or a combination of hardware and software) that operates on the patient dental data and generates dental appliance data based on (directed by) the practitioner-specific subset of textural rules. The dental appliance data may be used to fabricate one or more dental aligners that may correspond to various stages of dental treatment. In some examples, the dental appliance data may be used to predict, render, and display tooth positions of the patient corresponding to the stages of dental treatment. Through the displayed tooth positions, the clinician can assess each stage of the patient's treatment. In some cases, the clinician may modify the practitioner-specific subset of textural rules to change the outcome of any particular treatment stage.

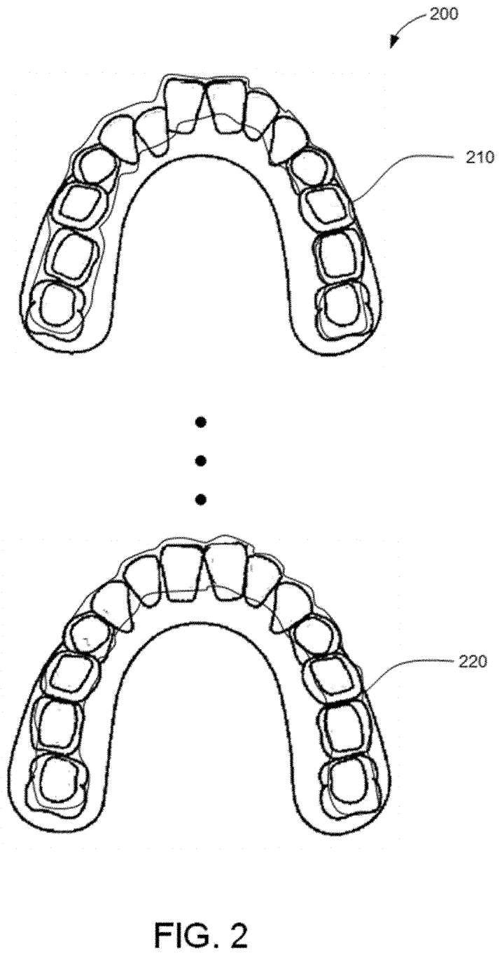

FIG. 2 shows a top view of clear dental aligners 200 that may be fabricated from the dental appliance data described with respect to FIG. 1. The clear dental aligners 200 may be used to provide orthodontic dental treatment to a patient. In some examples, a clinicians treatment protocol, as described by the clinician's rule, may be used to generate dental appliance data based on the patient's dental data. The orthodontic dental treatment may be implemented in stages, such that a different dental aligner may be associated with each treatment stage. The dental appliance data may describe any feasible number of stages, and therefore, any feasible number of dental aligners.

A first dental aligner 210 is shown. The first dental aligner 210 may correspond to a patient's initial tooth positions as determined by the patient dental data. The final dental aligner 220 may correspond to projected final tooth positions of the patient. Any feasible number of dental aligners may be fabricated (not shown for simplicity). For example, other dental aligners may be generated that correspond to different stages of dental treatment between the first dental aligner 210 and the final dental aligner 220.

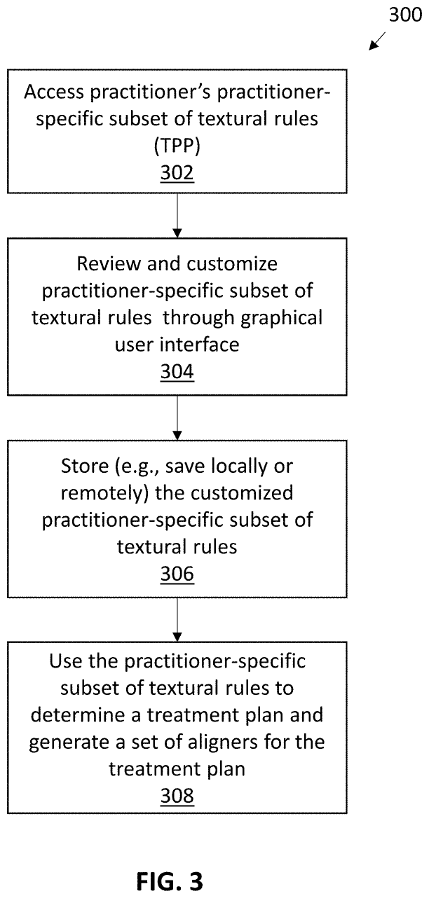

As described with respect to FIG. 1, a practitioner's practitioner-specific subset of textural rules may guide or affect an outcome of a dental treatment plan. In some cases, the clinician may want to modify their selected rules to change the outcome of the dental treatment. One example method for modifying a rule is described below in conjunction with FIG. 3.

FIG. 3 is a flowchart depicting an example method 300 for customizing a rule, in accordance with some examples. As described above with respect to FIGS. 1 and 2, a rule may describe a clinician's instructions for forming a dental treatment protocol. In some cases, the rule(s) may originate from another user, from a set of default rule parameters, or from an interview, questionnaire or the like between the clinician and a technician. Independent of the origination of the rule, the clinician may want to customize the rule to reflect the clinician's own preferences. Although described as being performed by a module, the method 300 may be performed by any feasible, hardware, software, combination of hardware and software, state machine, or the like. In some examples, the method 300 may be performed locally or performed remotely (e.g., through a cloud-based storage system) away from the clinician.

The method may begin at block 302 where the module accesses the clinician's rule. The clinician's rule may be a default rule, a rule from or based on another user's rule, or a rule provided to the clinician as a result of an interview, a questionnaire, a meeting, or any other feasible rule source. In some examples, the module may access the clinician's rule locally (e.g., through a local memory and/or a local storage device) or remotely through one or more cloud-based storage systems.

Next, in block 304, the clinician reviews and customizes the rule through a graphical user interface (GUI). For example, the rule may include one or more sections that describe particular characteristics of the clinician's dental protocol. As described above, the instructions associated with the rule may be written in a domain-specific treatment programming language that may not be easily understood, particularly by some dental clinicians. In block 304, the module may parse the rule and display one or more features and/or characteristics of the rule in a human-readable, clinician-friendly format on the GUI. That is, the GUI may show, display and/or describe some features and/or characteristics of the rule in an easily understood, clear language, in contrast to the domain-specific treatment programming language of the rule.

The rule features and/or characteristics may be displayed on the GUI along with a radio button or other user interactive feature. Using the interactive feature, the clinician can include or exclude the corresponding rule feature in the rule. In this manner, the clinician can customize the rule without having any prior knowledge of any domain-specific treatment programming language. Thus, the clinician may create a customized or personalized rule.

Next, in block 306, the customized rule and the practitioner's practitioner-specific subset of textural rules may be saved. The customized practitioner-specific subset of textural rules may be saved locally or remotely through one or more cloud-based storage systems. Next, in block 308, the clinician may use the customized practitioner-specific subset of textural rules to determine a patient's dental appliance data (e.g., a treatment plan). In some implementations, dental appliance data may be determined as described in U.S. patent application Ser. No. 16/399,834 entitled “Systems and Methods for Treatment Using Domain-Specific Treatment Protocols,” the contents of which are incorporated by reference as if set forth fully herein. In some cases, the dental appliance data may be used to fabricate aligner trays to provide orthodontic treatment. In some other cases, the dental appliance data may be used to determine predicted positions of a patient's teeth through different stages of orthodontic treatment. These predicted positions may be used to render images of the patient's teeth during treatment. The clinician may use the rendered images to evaluate the proposed treatment.

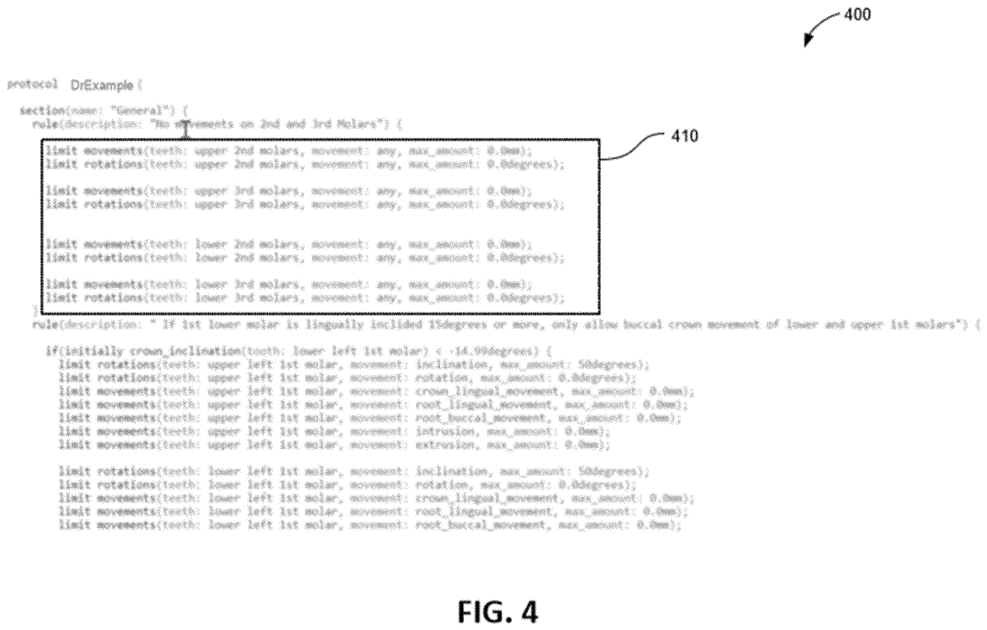

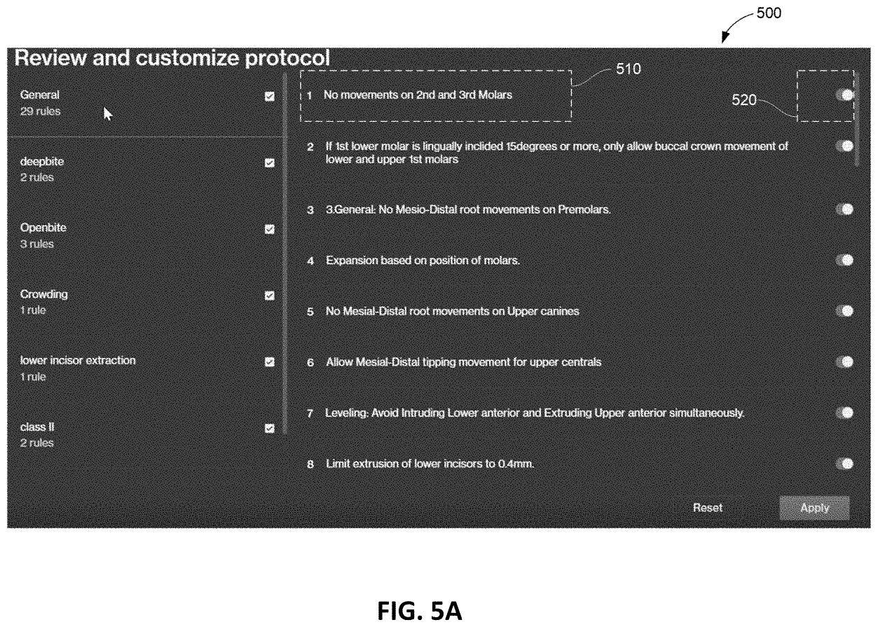

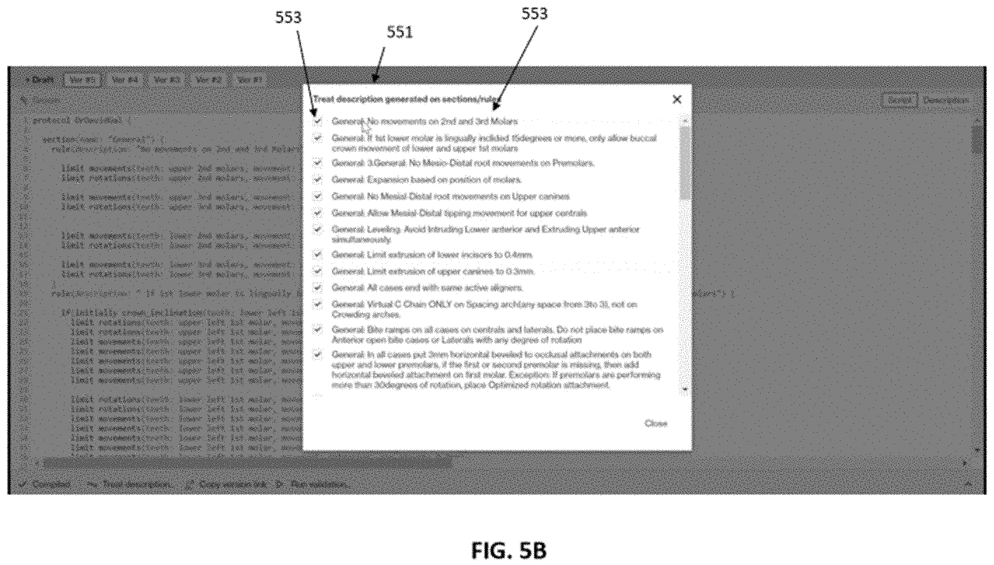

FIGS. 4 and 5A-5B illustrate certain aspects of the method as described herein. In particular, FIG. 4 shows one example portions of a rule and an example corresponding GUI display, showing the domain-specific treatment language corresponding to this example rule in accordance with the method 300. The FIGS. 4 and 5A-5B are exemplary and meant to explain the method 300 and are not intended to be limiting.

FIG. 4 shows an excerpt of a rule 400, in accordance with some examples. The rule 400 may be written in a domain-specific treatment programming language that may be difficult for some clinicians to understand. In some cases, the rule 400 may be a default rule, a rule from or based on another user's rule, or a rule provided to a clinician as a result of an interview, a questionnaire, a meeting, or any other feasible rule source.

Embedded within the rule 400 may be a plurality of sections and rules. Within the sections and rules, the domain-specific treatment programming language of the rule may describe various aspects of a clinician's dental protocol for performing dental treatment. For example, programming language that describes limiting molar movement of second and third molars may be included the portion 410 of the rule 400. In this example, eight lines of programming language may be used to describe limiting the molar movement of the second and third of a patient. The rule 400 may include other sections and rules not shown here for simplicity.

While there may be some clinicians that can easily comprehend the domain-specific treatment programming language of the rule, many other clinicians may find the programming language difficult to understand. Using the method 300 of FIG. 3, one or more sections of the rule may be parsed and displayed to the clinician in a human-readable, clinician-friendly format.

FIG. 5A shows a GUI 500 corresponding to a subset of rules similar to that of FIG. 4. This example of a GUI 500 may enable a clinician to select and include (or deselect and exclude) a rule in the practitioner's practitioner-specific subset of textural rules. The GUI 500 may display portions of a corresponding rule in a human-readable, clinician-friendly format, and/or in the domain-specific treatment language. Furthermore, the GUI 500 may include user-interactive features (selectable buttons, sliders, or the like) associated with the human-readable, clinician-friendly text. By interacting with the user-interactive features, the clinician may determine which protocol characteristics may be included or excluded from the practitioner's practitioner-specific subset of textural rules.

For example, the GUI 500 may display a rule characteristic of “no movements on 2^{nd} and 3^{rd} Molars” in section 510. The language in the section 510 may be textual, and describe in a clinician-friendly format; this language may correspond to one or more lines of the domain-specific treatment programming language of the rule (such as, for example, the lines in portion 410 of FIG. 4). Adjacent to the section 510, the GUI 500 may also include user-interactive features 520 to enable the clinician to include or exclude particular rules corresponding of the practitioner's practitioner-specific subset of textural rules.

By way of example, if the clinician wishes to implement “no movements on 2^{nd} and 3^{rd} molars,” then the clinician simply selects this feature by interacting with the user-interactive feature 520 corresponding to the “no movements on 2^{nd} and 3^{rd} molars.” On the other hand, if the clinician wishes to not implement “no movements on the 2^{nd} and 3^{rd} molars,” then the clinician can deselect this feature with the user interactive feature 520. Other rules may similarly be included or excluded in the practitioner's practitioner-specific subset of textural rules.

As a result of the clinician's selections through the GUI 500, the clinician may create a customized practitioner-specific subset of textural rules that may be used to generate treatment plans that may be used to fabricate dental appliances and/or display predicted tooth positions. Thus, the clinician, without having any prior knowledge of the domain-specific treatment programming language, may modify and customize a practitioner's practitioner-specific subset of textural rules for generating the treatment plan(s).

FIG. 5B shows another example of a user interface including a window 551 showing a menu or rules 553 (similar to that shown in FIG. 5A) and allowing direct user interface to select 553 among or between the rules displayed or displayable in the window. The rules may be organized (e.g., ranked, grouped, etc.) based on the instructions associated with the rules. As shown in FIG. 5A the rules may be organized by category (e.g., “General”, “Deep bite”, “Crowding”, “Opening”, “lower incisor extraction”, “Class II”, etc.). In some cases the rules may be organized by a score indicating how frequently they are used by others. The rules may be organized and/or ranked by geographic use and/or by association with one or more opinion leaders. The method or apparatus may pre-select which rules to list in the menu as described above, including displaying or including rules for the practitioner to select from in building the practitioner's practitioner-specific subset of textural rules based on one or more characteristic of the practitioner and/or response to an inquiry related to the practitioner.

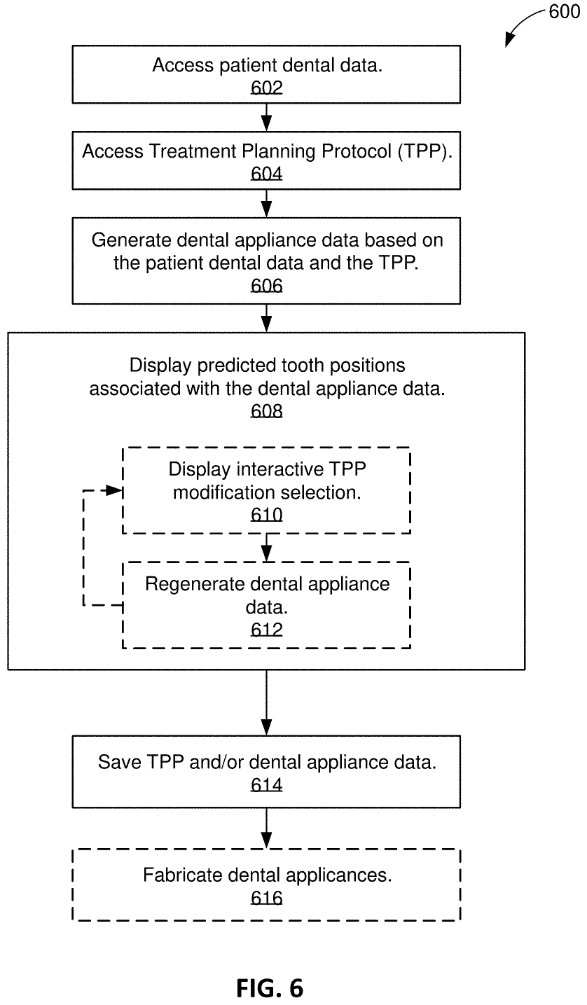

In some examples, the clinician may want to modify the practitioner-specific subset of textural rules in a more interactive manner that can illustrate how changes in the practitioner-specific subset of textural rules may affect the outcome of dental treatments. One such method is described below with respect to FIG. 6.

FIG. 6 is a flowchart depicting an example method 600 for generating dental appliance data, in accordance with some examples. Although described as being performed by a module, the method 600 may be performed by any feasible, hardware, software, combination of hardware and software, state machine, or the like.

The method 600 may begin in block 602 where the module accesses (retrieves) patient dental data. The patient dental data may include dental scan data, patient demographic data, or any other relevant patient data relating to the patient's dental and/or orthodontic physiology. The patient dental data may be stored locally (with respect to the module) or remotely, for example, using a cloud-based storage system. Next, in block 604, the module may access (retrieve) the practitioner's practitioner-specific subset of textural rules. The practitioner's practitioner-specific subset of textural rules may be a default set of rules, a set of rules from or based on another user's set of rules, a set of rules provided to the clinician as a result of an interview, a questionnaire, a meeting, or any other feasible rule source. The practitioner's practitioner-specific subset of textural rules may be stored locally or remotely, for example, using a cloud-based storage system. In some examples, the practitioner's practitioner-specific subset of textural rules may have been generated in accordance with the method 300 of FIG. 3.

Next, in block 606, the module may generate dental appliance data based on the patient dental data and the practitioner-specific subset of textural rules. For example, the module may generate dental appliance data by applying the dental protocols described by the practitioner's practitioner-specific subset of textural rules on the patient's dental data. In some examples, the module may generate multiple stages of dental appliance data in accordance with one or more dental treatment stages. In some cases, each stage of the dental treatment may correspond to a different dental aligner.

Next, in block 608, the module displays predicted tooth positions associated with the dental appliance data. In some cases, the module can predict tooth movement and positions from the dental appliance data. Since the dental appliance data may correspond to multiple treatment stages, the module may display a different image for each treatment stage. The clinician may use the displayed images to determine if the practitioner-specific subset of textural rules used to generate the dental appliance data provides a desired dental treatment outcome.

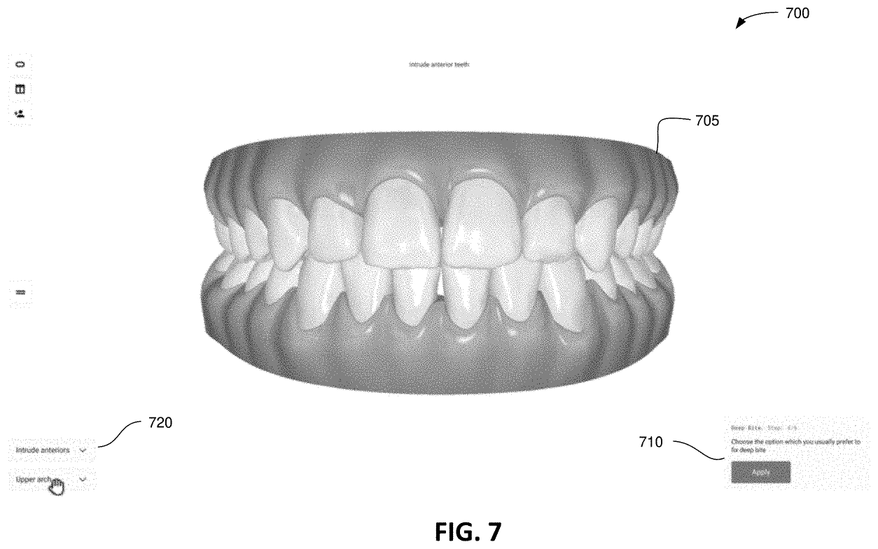

Next, in block 610, the module may optionally display one or more interactive rule modification selections. In some cases, the clinician may optionally interact with the display to modify the practitioner-specific subset of textural rules. For example, if the desired outcome is not achieved (as shown in the displayed images in block 608), then the clinician may interact with the GUI to modify one or more of the rules from the practitioner-specific subset of textural rules. (An example interactive display is described below with respect to FIG. 7.) In some cases, the GUI may present dialog boxes and the like to enable the clinician to modify some or all of the rules (and parameters) that can affect teeth movement, and therefore, affect the dental treatment outcome.

Next, in block 612, the module may optionally regenerate the dental appliance data. For example, if the practitioner has modified one or more rule parameters of the practitioner's practitioner-specific subset of textural rules, then the module can regenerate the dental appliance data and (with respect to block 608) the module may display the predicted tooth positions associated with the regenerated dental appliance data. The clinician may review the displayed predicted tooth positions (as displayed in block 608) to review the dental treatment outcome. In some examples, the blocks 610 and 612 may be repeated any number of times until, for example, the clinician approves of the dental treatment outcome.

Next, in block 614, the module may save the modified practitioner-specific subset of textural rules and/or the dental appliance data. Since the practitioner-specific subset of textural rules may have been modified in block 608, the module may enable the clinician to save the modified rule. The modified practitioner-specific subset of textural rules may be used by the clinician as a custom rule or set of textural rules that may be used with other patient's and/or may be shared with other clinicians. Furthermore, the dental appliance data that resulted from the use of the practitioner's practitioner-specific subset of textural rules may also be saved. The practitioner-specific subset of textural rules and the dental appliance data may be saved locally or remotely, for example, using a cloud-based storage system.

Next, in block 616 the dental appliances may be fabricated. In this optional step, the dental aligners corresponding to the dental appliance data may be fabricated. The dental aligners may be used by the patient to receive the dental therapy as determined by the clinician.

In some examples, after modifying and saving the practitioner-specific subset of textural rules, the clinician may process other patient's dental data with the modified practitioner-specific subset of textural rules. In this manner, the clinician may “test” the modified practitioner-specific subset of textural rules with a variety of different patient data in order to determine whether the modified practitioner-specific subset of textural rules will be effective in a variety of cases, and not just a special case of one patient.

FIG. 7 shows an example GUI 700 including interactive rule modification selections, in accordance with some examples. As described above with respect to FIG. 6, the GUI 700 may display predicted tooth positions that may correspond to dental appliance data. For example, the predicted tooth positions may be associated with any dental aligner data that is associated with a dental treatment directed or guided by a practitioner's practitioner-specific subset of textural rules. Through the GUI, the clinician may modify rule parameters to alter the dental treatment outcome. The GUI 700 may display a predicted tooth position 705 that depicts possible tooth positions associated with at least one stage of the dental treatment of the patient.

As shown, the GUI 700 may also include a selection prompt 710. The selection prompt 710 may indicate to the clinician that an aspect of the treatment illustrated on the GUI 700 may be affected by modifying one or more parameters of the practitioner-specific subset of textural rules. The selection prompt 710 may draw attention to rule selections 720 and prompt the clinician to select and/or modify one or more practitioner-specific subset of textural rules parameters through the rule selections 720. The selection choice may be applied and/or confirmed through the selection prompt 710.

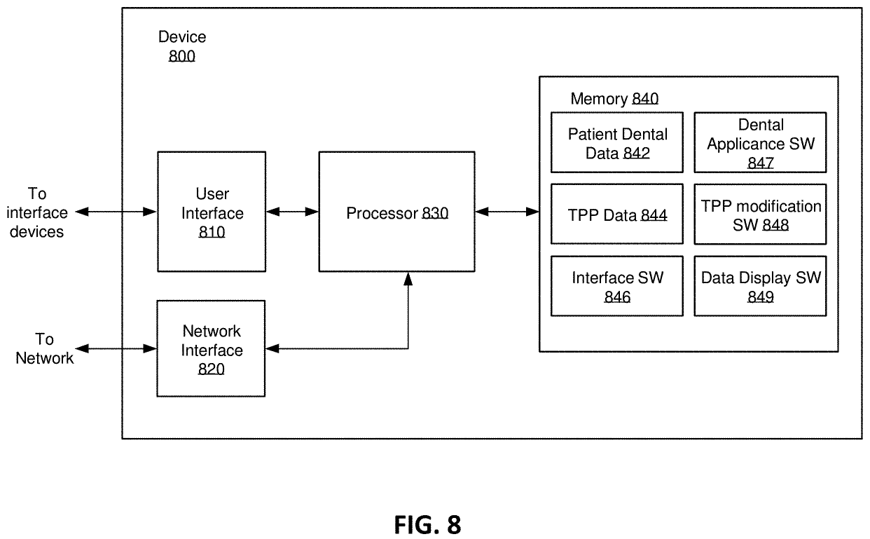

FIG. 8 shows a block diagram of a device 800 that may be configured to perform any of the operations described herein. The device 800 may include a user interface 810, a network interface 820, a processor 830, and a memory 840. The user interface 810 may be coupled to any feasible user interface (not shown) including, but not limited to, a display, a keyboard, a mouse or other pointing device, a touch screen, and so on. The network interface 820, which may be coupled to a network (not shown), may transmit signals to and receive signals from other wired or wireless devices, including remote (e.g., cloud-based) storage devices.

The one or more processors 830, which is/are also coupled to the user interface 810, the network interface 820, and the memory 840, may be any one or more suitable processors capable of executing scripts or instructions of one or more software programs stored in the device 800 (such as within the memory 840). In some examples, the memory 840 may be implemented remotely, for example, in a cloud-based storage device.

The memory 840 may include (store) patient dental data 842. As described herein, patient dental data may include patient dental scan data, patient demographic data, patient physiological data, or any other feasible patient data that may be useful in determining a patient's dental therapy.

The memory 840 may also include a practitioner-specific subset of textural rules data 844. As described herein, the rule data may be used by another module to determine a patient's dental therapy which may include a patient's dental appliance data.

The memory 840 may also include a non-transitory computer-readable storage medium (e.g., one or more nonvolatile memory elements, such as EPROM, EEPROM, Flash memory, a hard drive, etc.) that may store the following software modules: an interface software (SW) module 846 to control interfaces, including, for example, the user interface 810, and the network interface 820; a dental appliance SW module 847 to generate a patient's dental appliance data; a rule modification SW module 848 to modify practitioner-specific subset of textural rules; and a data display SW module 849 to predict and display tooth movement. Each software module includes program instructions that, when executed by the processor 830, may cause the device 800 to perform the corresponding function(s). Thus, the non-transitory computer-readable storage medium of memory 840 may include instructions for performing all or a portion of the operations described herein.

The modules of the apparatus 800 may include one or more engines and datastores. A computer system can be implemented as an engine, as part of an engine or through multiple engines. As used herein, an engine includes one or more processors or a portion thereof. A portion of one or more processors can include some portion of hardware less than all of the hardware comprising any given one or more processors, such as a subset of registers, the portion of the processor dedicated to one or more threads of a multi-threaded processor, a time slice during which the processor is wholly or partially dedicated to carrying out part of the engine's functionality, or the like. As such, a first engine and a second engine can have one or more dedicated processors or a first engine and a second engine can share one or more processors with one another or other engines. Depending upon implementation-specific or other considerations, an engine can be centralized or its functionality distributed. An engine can include hardware, firmware, or software embodied in a computer-readable medium for execution by the processor. The processor transforms data into new data using implemented data structures and methods, such as is described with reference to the figures herein.

The engines described herein, or the engines through which the systems and devices described herein can be implemented, can be cloud-based engines. As used herein, a cloud-based engine is an engine that can run applications and/or functionalities using a cloud-based computing system. All or portions of the applications and/or functionalities can be distributed across multiple computing devices, and need not be restricted to only one computing device. In some embodiments, the cloud-based engines can execute functionalities and/or modules that end users access through a web browser or container application without having the functionalities and/or modules installed locally on the end-users' computing devices.

As used herein, datastores are intended to include repositories having any applicable organization of data, including tables, comma-separated values (CSV) files, traditional databases (e.g., SQL), or other applicable known or convenient organizational formats. Datastores can be implemented, for example, as software embodied in a physical computer-readable medium on a specific-purpose machine, in firmware, in hardware, in a combination thereof, or in an applicable known or convenient device or system. Datastore-associated components, such as database interfaces, can be considered “part of” a datastore, part of some other system component, or a combination thereof, though the physical location and other characteristics of datastore-associated components is not critical for an understanding of the techniques described herein.

Datastores can include data structures. As used herein, a data structure is associated with a particular way of storing and organizing data in a computer so that it can be used efficiently within a given context. Data structures are generally based on the ability of a computer to fetch and store data at any place in its memory, specified by an address, a bit string that can be itself stored in memory and manipulated by the program. Thus, some data structures are based on computing the addresses of data items with arithmetic operations; while other data structures are based on storing addresses of data items within the structure itself. Many data structures use both principles, sometimes combined in non-trivial ways. The implementation of a data structure usually entails writing a set of procedures that create and manipulate instances of that structure. The datastores, described herein, can be cloud-based datastores. A cloud-based datastore is a datastore that is compatible with cloud-based computing systems and engines.

The processor 830 may execute the interface SW module 846 to transmit and receive data through the user interface 810 and/or the network interface 820. For example, execution of the interface SW module 846 may enable the processor 830 to display images on a display device (e.g., GUI) and/or receive data, such as user input data (keyboard data, scan data, touch pad data, and the like) through the user interface 810. Execution of the interface SW module 846 may also enable the processor 830 to transmit and receive data through the network interface 820. For example, the processor 830 may transmit and receive data that may be stored on a separate or remote data storage device (not shown) through the internet or any other feasible network. In some examples, the processor 830 may transmit dental appliance data through the network interface 820 to enable fabrication of the corresponding dental appliances.

The processor 830 may execute the dental appliance SW module 847 to generate a patient's dental appliance data based on the patient dental data 842 and the rule data 844. The dental appliance data may be used to fabricate one or more dental aligners to provide a dental treatment.

The processor 830 may execute the rule modification SW module 848 to modify or alter rule parameters such as rule data 844 stored in the memory 840. In some examples, execution of the rule modification SW module 848 may enable the processor 830 to display and modify a rule of the practitioner-specific subset of textural rules as described with respect to FIG. 3. In some other examples, execution of the rule modification SW module 848 may enable the processor 830 to display and modify a rule or practitioner-specific subset of textural rules as described above.

The processor 830 may execute the data display SW module 849 to generate data that may be displayed on a user's display. For example, execution of the data display SW module 849 may enable the processor 830 to render predicted tooth positions (based on patient dental data) as well as interactive screens to prompt the clinician regarding changes to the clinician's rule.

The medical treatment planning may allow users to create patient-customized treatment protocols. For example, orthodontic treatment planning allows users to create patient-customized treatment protocols. Such protocols may include rules for final positioning, staging, attachment and aligner features, and may define conditional behaviors depending on the treatment goals, the initial or final position of teeth, or the existence of various clinical conditions. Manual orthodontic treatment planning may be slow and complicated, even when assisted by orthodontic treatment planning algorithms, which typically use simple parameter files. This is particularly true when users require higher degree of customization, which may be accommodated by either applying their protocols manually (which can be labor-intensive and may result in inconsistent results), or by coding special rules in a shared code base (which may result in long validation cycles).

The present disclosure is related to systems, methods, computing device readable media, and devices that solve technical problems related to treatment planning including, in particular, orthodontic treatment planning and/or technical problems related to fabrication of dental appliances (e.g., aligners) as part of an orthodontic treatment plan. Automated agents (including those that use machine learning models) may be used to aid in forming, modifying and processing treatment templates, which may be encoded in a domain-specific orthodontic treatment language. In some implementations, the automated agents described herein provide treatment templates, which may be converted into orthodontic treatment planning instructions, and used with one or more sets of patient data (e.g., scans of a patient's teeth) to generate one or more orthodontic treatment plans. Orthodontic treatment plans may include descriptions or instructions to fabricate dental appliances, such as dental aligners.

### Example Structures and Systems

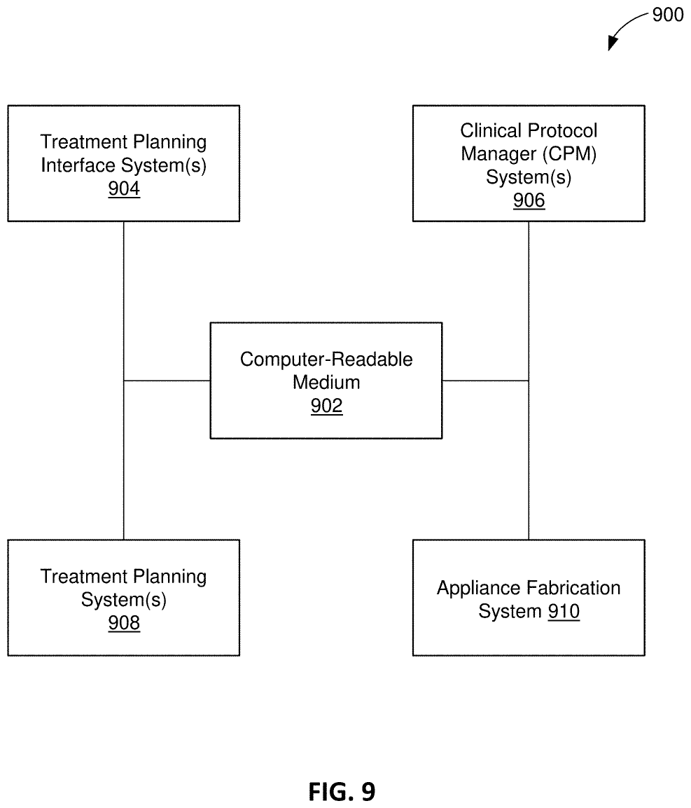

FIG. 9 is a diagram showing an example of systems in a device planning environment 900. The device planning environment 900 includes a computer-readable medium 902, treatment planning interface system(s) 904, a clinical protocol manager (CPM) system(s) 906, treatment planning system(s) 908, and appliance fabrication system(s) 910. One or more of the components (including modules) of the orthodontic treatment planning system 900 may be coupled to one another (e.g., through the example couplings shown) or to modules not explicitly shown in FIG. 9. The computer-readable medium 902 may include any computer-readable medium, including without limitation a bus, a wired network, a wireless network, or some combination thereof.

A computer system can be implemented as an engine, as part of an engine or through multiple engines. As used herein, an engine includes one or more processors or a portion thereof. A portion of one or more processors can include some portion of hardware less than all of the hardware comprising any given one or more processors, such as a subset of registers, the portion of the processor dedicated to one or more threads of a multi-threaded processor, a time slice during which the processor is wholly or partially dedicated to carrying out part of the engine's functionality, or the like. As such, a first engine and a second engine can have one or more dedicated processors or a first engine and a second engine can share one or more processors with one another or other engines. Depending upon implementation-specific or other considerations, an engine can be centralized or its functionality distributed. An engine can include hardware, firmware, or software embodied in a computer-readable medium for execution by the processor. The processor transforms data into new data using implemented data structures and methods, such as is described with reference to the figures herein.

The engines described herein, or the engines through which the systems and devices described herein can be implemented, can be cloud-based engines. As used herein, a cloud-based engine is an engine that can run applications and/or functionalities using a cloud-based computing system. All or portions of the applications and/or functionalities can be distributed across multiple computing devices, and need not be restricted to only one computing device. In some embodiments, the cloud-based engines can execute functionalities and/or modules that end users access through a web browser or container application without having the functionalities and/or modules installed locally on the end-users' computing devices.

As used herein, datastores (e.g., data bases or other warehouses of data) are intended to include repositories having any applicable organization of data, including tables, comma-separated values (CSV) files, traditional databases (e.g., SQL), or other applicable known or convenient organizational formats. Datastores can be implemented, for example, as software embodied in a physical computer-readable medium on a specific-purpose machine, in firmware, in hardware, in a combination thereof, or in an applicable known or convenient device or system. Datastore-associated components, such as database interfaces, can be considered “part of” a datastore, part of some other system component, or a combination thereof, though the physical location and other characteristics of datastore-associated components is not critical for an understanding of the techniques described herein.

Datastores can include data structures. As used herein, a data structure is associated with a particular way of storing and organizing data in a computer so that it can be used efficiently within a given context. Data structures are generally based on the ability of a computer to fetch and store data at any place in its memory, specified by an address, a bit string that can be itself stored in memory and manipulated by the program. Thus, some data structures are based on computing the addresses of data items with arithmetic operations; while other data structures are based on storing addresses of data items within the structure itself. Many data structures use both principles, sometimes combined in non-trivial ways. The implementation of a data structure usually entails writing a set of procedures that create and manipulate instances of that structure. The datastores, described herein, can be cloud-based datastores. A cloud-based datastore is a datastore that is compatible with cloud-based computing systems and engines.

The treatment planning interface system(s) 904 may include one or more computer systems configured to interact with users and provide users with the ability to manage treatment plans for patients. A “user,” in this context, may refer to any individual who can access and/or use the treatment planning interface system(s) 904, and can include any medical professional, including dentists, orthodontists, podiatrists, medical doctors, surgeons, clinicians, etc.

In some implementations, the treatment planning interface system(s) 904 includes engines to gather patient data related to patients who are to be treated according to a treatment plan.

“Patient data,” as used herein, may include data related to a patient. Patient data may include representations of anatomical information, such as information about specific portions of the human body to be treated. Examples of anatomical information include representations of a patient's dentition, bones, organs, etc. at a specific time. Patient data may represent anatomical information before, during, or after a treatment plan. As examples, patient data may represent the state and/or intended state of a patient's dentition before, during, or after orthodontic or restorative treatment plans. Patient data may be captured using a variety of techniques, including from a scan, digitized impression, etc. of the patient's anatomy.

A “treatment plan,” as used herein, may include a set of instructions to treat a medical condition. A treatment plan may specify, without limitation treatment goals, specific appliances used to implement the goals, milestones to measure progress, and other information, such as treatment length and/or treatment costs. As noted herein, in some implementations, the treatment planning interface system(s) 904 provides a user with an orthodontic treatment plan to treat malocclusions of teeth. The treatment planning interface system(s) 904 may also provide users with restorative treatment plans for a patient's dentition and other types of medical treatment plans to address medical conditions patients may have. In some implementations, a treatment plan may include an automated and/or real-time treatment plan, such as the treatment plans described in U.S. patent application Ser. No. 16/178,491, entitled “Automated Treatment Planning,” the contents of which are incorporated by reference as if set forth fully herein. A treatment plan may also include treatment instructions provided by a treatment technician, such as a treatment technician who provides the treatment plan to the user of the treatment planning interface system(s) 904 through the computer-readable medium 902.

In various implementations, the treatment planning interface system(s) 904 is configured to allow a user to visualize, interact with, and/or fabricate appliances that implement a treatment plan. As an example, the treatment planning interface system(s) 904 may provide a user with a user interface that displays virtual representations of orthodontic appliances that move a patient's teeth from an initial position toward a final position to correct malocclusions of teeth. The treatment planning interface system(s) 904 can similarly display representations of restorative appliances and/or other medical appliances. The treatment planning interface system(s) 904 may allow a user to modify appliances through a UI supported thereon. In various implementations, the treatment planning interface system(s) 904 allows a user to fabricate appliances through, e.g., the appliance fabrication system(s) 910. (It is noted the appliance fabrication system(s) 910 may but need not be remote to the treatment planning interface system(s) 904 and can be located proximate to the treatment planning interface system(s) 904.)

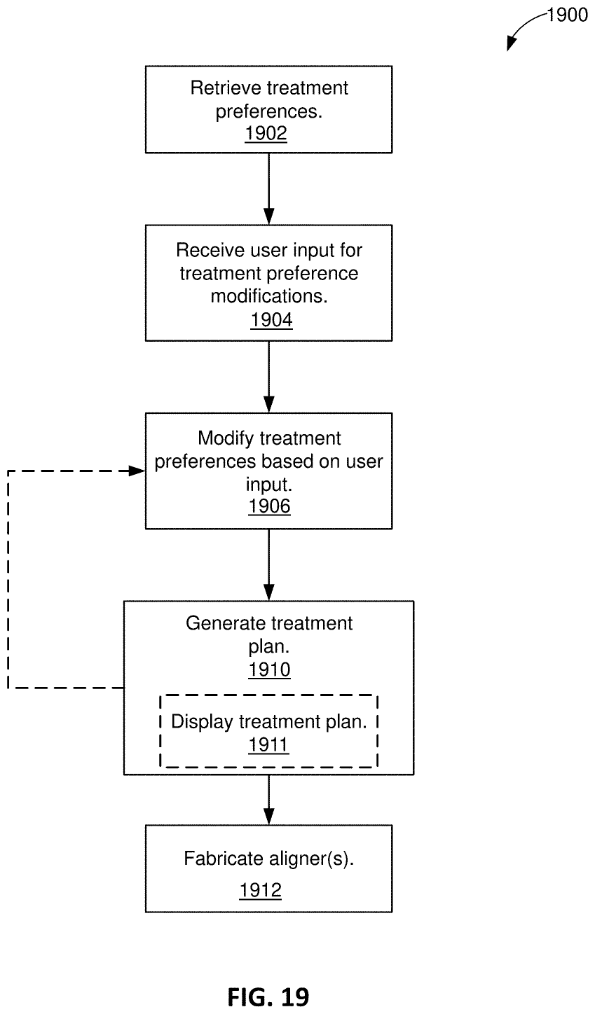

The treatment planning interface system(s) 904 may be configured to provide a user with UIs that allow the user to discuss treatment plans with patients. As an example, the treatment planning interface system(s) 904 may display to the user portions of patient data (e.g., depictions of a condition to be treated) as well as treatment options to correct a condition. The treatment planning interface system(s) 904 may display potential appliances that are prescribed to implement the treatment plan. As an example, the treatment planning interface system(s) 904 may display to the user a series of orthodontic appliances that are configured to move a patient's dentition from a first position toward a target position in accordance with an orthodontic treatment plan. The treatment planning interface system(s) 904 may further be configured to depict the effects of specific appliances at various stages of a treatment plan. In some implementations, the treatment planning interface system(s) 904 may implement a method of using a treatment protocol to generate a treatment plan as shown in FIG. 19.

The treatment planning interface system(s) 905 may be configured to allow a user to interact with a treatment plan. In some implementations, the treatment planning interface system(s) 904 allows a user to specify treatment preferences. “Treatment preferences,” as used herein, may include specific treatment options and/or treatment tools that a user prefers when treating a condition. Treatment preferences may include clinical settings, treatment goals, appliance attributes, preferred ranges of movement, specific stages to implement a specific procedure, etc. Examples of clinical settings in an orthodontic context include allowing or disallowing a type of treatment, use of various types of movements on specific teeth (e.g., molars), use of specific procedures (e.g., interproximal reduction (IPR)), use of orthodontic attachments on specific teeth, etc. Examples of treatment goals in an orthodontic context include lengths/costs of treatments, specific intended final and/or intermediate positions of teeth, etc. Example ranges of movement in an orthodontic context include specific distances and/or angles teeth are to move over various stages of treatment and/or specific forces to be put on teeth over various stages of treatment. Specific stages to implement a specific procedure include, for instance in the orthodontic context, a specific treatment stage to implement attachments, hooks, bite ramps and/or to perform procedures such as surgery or interproximal reduction.

As discussed further herein, the treatment planning interface system(s) 904 may be configured to provide users with customized GUI elements based on treatment templates that structure their treatment preferences in a manner that is convenient to them. Customized GUI elements may include forms, text boxes, UI buttons, selectable UI elements, etc.). In some implementations, customized GUI elements may list treatment preferences and provide a user with the ability to accept, deny, and/or modify treatment preferences. Customized GUI elements may provide the ability to accept or deny parts of at treatment plan and/or modify portions of a treatment plan. In some implementations, a user's customized GUI elements provide the ability to modify parts of an appliance recommended for a treatment plan. For instance, a treatment-related UI element may provide the ability to modify force systems, velocities of tooth movement, angles and/or orientations of parts of aligners, crowns, veneers, etc. that are implemented at specific stages of an orthodontic or restorative treatment plan.

“Treatment templates,” as used herein, may include structured data expressed in “treatment domain-specific protocols.” (In some examples, treatment templates are generated by the CPM system(s) 906, stored in datastores on the treatment planning system(s) 908, and parsed by engines on the treatment planning system(s) 908 that create customized GUI elements on the treatment planning interface system(s) 904.)

“Treatment domain-specific protocols,” as used herein, may include computer languages, runtime objects (e.g., applications, processes, etc.), interpreted items (e.g., executed scripts), etc. that are specialized to treatment planning. Treatment domain-specific protocols may include attributes that are specialized to patient data and/or the gathering thereof, attributes that are specialized to description and/or interaction with treatment plans, and attributes that are specialized to appliances used to implement a treatment plan. The present disclosure provides a detailed example of orthodontic domain-specific protocols. It is noted the examples herein may apply to restorative and/or dental domain-specific protocols and other medical domain-specific protocols.

In some implementations, treatment templates include customized graphical user interface (GUI) elements. Customized GUI elements may be generated using treatment domain-specific protocols. As noted herein, the treatment templates for a user may be customized based on a template library of treatment templates for other users. As an example, a treatment template for a user may be derived from and/or otherwise based on a treatment template of another user (e.g., the treatment preferences in that treatment template may be derived from and/or otherwise based on treatment preferences of another user). Public templates may provide the basis of deriving treatment preferences of other users. Private templates may provide a basis of deriving treatment preferences of a specific user. Additionally, customized GUI elements may be automatically generated during execution of applications and/or processes on the treatment planning interface system(s) 904. Customized GUI elements may operate to display attributes of treatment plans that are relevant to a specific user.

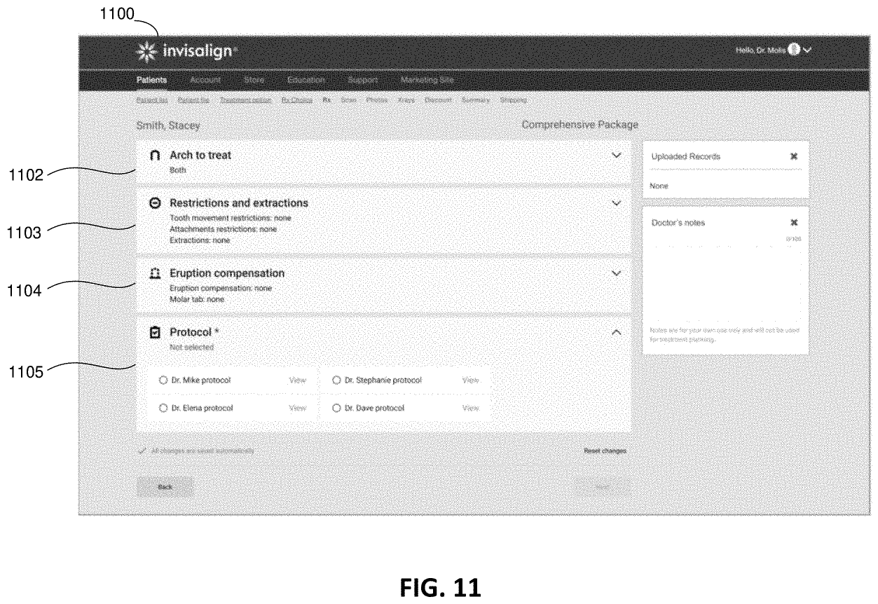

The CPM system(s) 906 may include one or more computer systems configured to create treatment templates using treatment domain-specific protocols. In some implementations, the CPM system(s) 906 are operated by CPM technicians, who may, but need not, be remote to users of the treatment planning interface system(s) 904. The CPM system(s) 906 may also be operated by automated agents. The CPM system(s) 906 may include tools to create treatment templates for specific users based on unstructured representations of treatment preferences of those users. In some implementations, the CPM system(s) 906 are configured to obtain past treatment preferences for users through telephonic interviews, emails, notes memorializing discussions, etc. The CPM system(s) 906 may provide technicians with editing tools to structure treatment preferences in a manner that can be organized for a treatment domain-specific protocol. In various implementations, the CPM system(s) 906 are configured to support creating and editing of treatment domain-specific protocols. As an example, the CPM system(s) 906 may be configured to allow technicians to create and/or edit treatment domain-specific scripts that structure treatment preferences for a specific user. An example flowchart of a method of creating or editing of treatment domain-specific protocols is shown in FIG. 19. Example screen capture of editing tools supported by the CPM system(s) 906 are shown in FIG. 11.

Additionally, the CPM system(s) 906 may provide validation tools to validate treatment domain-specific protocols to ensure the treatment domain-specific protocols are accurate or otherwise in line with treatment preferences. As an example, the CPM system(s) 2806 may provide a visual depiction of how specific treatment domain-specific protocols would appear in treatment planning software. As noted herein, the CPM system(s) 906 may employ one or more validation metrics to quantify validation. Examples of validation metrics that may be relevant to an orthodontic context include arch expansion metrics per quadrant, overjet metrics, overbite metrics, interincisal angle metrics, and/or flags if a treatment plan conforms with minimal or threshold root movement protocols.

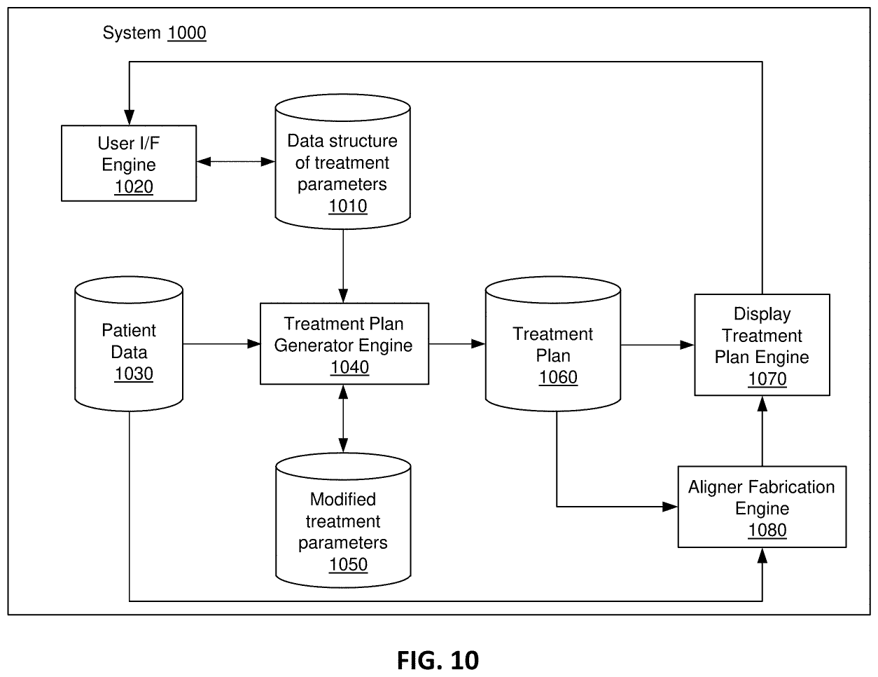

The CPM system(s) 906 may include one or more elements of the system 1000 shown in FIG. 10.

The treatment planning system(s) 908 may include one or more computer systems configured to provide treatment plans to the treatment planning interface system(s) 904. The treatment planning system(s) 908 may receive patient data and the treatment preferences relevant to a user. The treatment planning system(s) 908 may further provide treatment plans for the patient data that accommodate the treatment preferences relevant to the user. The treatment planning system(s) 908 may implement automated and/or real-time treatment planning as referenced further herein.

The treatment planning system(s) 908 may include one or more engines configured to provide treatment plans to the treatment planning interface system(s) 904. The treatment planning system(s) 908 may receive patient data and the treatment preferences relevant to a user. The treatment planning system(s) 908 may further provide treatment plans for the patient data that accommodate the treatment preferences relevant to the user. In various implementations, the treatment planning system(s) 908 identify and/or calculate treatment plans with instructions treat medical conditions. The treatment plans may specify treatment goals, specific outcomes, intermediate outcomes, and/or recommended appliances used to achieve goals/outcomes. The treatment plan may also include treatment lengths and/or milestones. In various implementations, the treatment planning system(s) 908 calculate orthodontic treatment plans to treat malocclusions of teeth, restorative treatment plans for a patient's dentition, medical treatment plans, etc. The treatment plan may comprise automated and/or real-time elements and may include techniques described in U.S. patent application Ser. No. 16/178,491, entitled “Automated Treatment Planning.” In various implementations, the treatment planning system(s) 908 are managed by treatment technicians. As noted herein, the treatment plans may accommodate patient data in light of treatment preferences of users.

The treatment planning system(s) 908 may include engines that allow users of the treatment planning interface system(s) 904 to visualize, interact with, and/or fabricate appliances that implement a treatment plan. The treatment planning system(s) 908 may support UIs that display virtual representations of orthodontic appliances that move a patient's teeth from an initial position toward a final position to correct malocclusions of teeth. The treatment planning system(s) 908 can similarly include engines that configure the treatment planning interface system(s) 904 to display representations of restorative appliances and/or other medical appliances. The treatment planning system(s) 908 may support fabrication of appliances through, e.g., the appliance fabrication system(s) 910.

In some implementations, the treatment planning system(s) 908 provide customized GUIs that allow the user to discuss treatment plans with patients. The treatment planning system(s) 908 may render patient data, conditions to be treated, and/or treatment options for display on the treatment planning interface system(s) 904. The treatment planning system(s) 908 may render potential appliances that are prescribed to implement a treatment plan (e.g., series of orthodontic appliances that are configured to move a patient's dentition from a first position toward a target position in accordance with an orthodontic treatment plan; effects of specific appliances at various stages of a treatment plan, etc.). In some implementations, the treatment planning system(s) 908 supports a method of using a treatment protocol to generate a treatment plan as shown in FIG. 19.

The treatment planning system(s) 908 may include engines to support user interaction with treatment plans. The treatment planning system(s) 908 may use treatment preferences, including those generated in treatment domain-specific protocols by the CPM system(s) 906. In various implementations, the treatment planning system(s) 908 provide treatment templates to the treatment planning interface system(s) 904 that structure users' treatment preferences in a manner that is convenient to them. As noted herein, treatment templates may include structured data, UI elements (forms, text boxes, UI buttons, selectable UI elements, etc.), etc.

The treatment planning system(s) 908 may include one or more datastores configured to store treatment templates expressed according to treatment domain-specific protocols. The treatment planning system(s) 908 may further include one or more processing engines to process, e.g., parse, the treatment templates to form customized GUI elements on the treatment planning interface system(s) 904. As noted herein, the processing engines may convert the treatment templates into scripts or other runtime elements in order to support the customized GUI elements on the treatment planning interface system(s) 904. As noted herein, the treatment templates may have been created and/or validated by the CPM system(s) 906.

In some implementations, the treatment planning system(s) 908 provides the treatment planning interface system(s) 904 with customized GUI elements that are generated using treatment domain-specific protocols. The customized GUI elements may be based on treatment templates, which, for a user may be customized based on a template library of treatment templates for other users. The treatment templates may comprise public and/or private treatment. In some implementations, the treatment planning system(s) 908 generates customized GUI elements for display by applications and/or processes on the treatment planning interface system(s) 904. Customized GUI elements may operate to display attributes of treatment plans that are relevant to a specific user.

The appliance fabrication system(s) 910 may include one or more computer systems configured to fabricate appliances. As discussed herein, examples of appliances to be fabricated include dental as well as non-dental appliances. Examples of dental appliances include aligners, other polymeric dental appliances, crowns, veneers, bridges, retainers, dental surgical guides, etc. Examples of non-dental appliances include orthotic devices, hearing aids, surgical guides, medical implants, etc.

The appliance fabrication system(s) 910 may comprise thermoforming systems configured to indirectly and/or directly form appliances. The appliance fabrication system(s) 910 may implement instructions to indirectly fabricate appliances. As an example, the appliance fabrication system(s) 910 may be configured to thermoform appliances over a positive or negative mold. Indirect fabrication of a dental appliance can involve one or more of the following steps: producing a positive or negative mold of the patient's dentition in a target arrangement (e.g., by additive manufacturing, milling, etc.), thermoforming one or more sheets of material over the mold in order to generate an appliance shell, forming one or more structures in the shell (e.g., by cutting, etching, etc.), and/or coupling one or more components to the shell (e.g., by extrusion, additive manufacturing, spraying, thermoforming, adhesives, bonding, fasteners, etc.). Optionally, one or more auxiliary appliance components as described herein (e.g., elastics, wires, springs, bars, arch expanders, palatal expanders, twin blocks, occlusal blocks, bite ramps, mandibular advancement splints, bite plates, pontics, hooks, brackets, headgear tubes, bumper tubes, palatal bars, frameworks, pin-and-tube apparatuses, buccal shields, buccinator bows, wire shields, lingual flanges and pads, lip pads or bumpers, protrusions, divots, etc.) are formed separately from and coupled to the appliance shell (e.g., via adhesives, bonding, fasteners, mounting features, etc.) after the shell has been fabricated.

The appliance fabrication system(s) 910 may comprise direct fabrication systems configured to directly fabricate appliances. As an example, the appliance fabrication system(s) 910 may include computer systems configured to use additive manufacturing techniques (also referred to herein as “3D printing”) or subtractive manufacturing techniques (e.g., milling). In some embodiments, direct fabrication involves forming an object (e.g., an orthodontic appliance or a portion thereof) without using a physical template (e.g., mold, mask etc.) to define the object geometry. Additive manufacturing techniques can include: (1) vat photopolymerization (e.g., stereolithography), in which an object is constructed layer by layer from a vat of liquid photopolymer resin; (2) material jetting, in which material is jetted onto a build platform using either a continuous or drop on demand (DOD) approach; (3) binder jetting, in which alternating layers of a build material (e.g., a powder-based material) and a binding material (e.g., a liquid binder) are deposited by a print head; (4) fused deposition modeling (FDM), in which material is drawn though a nozzle, heated, and deposited layer by layer; (5) powder bed fusion, including but not limited to direct metal laser sintering (DMLS), electron beam melting (EBM), selective heat sintering (SHS), selective laser melting (SLM), and selective laser sintering (SLS); (6) sheet lamination, including but not limited to laminated object manufacturing (LOM) and ultrasonic additive manufacturing (UAM); and (7) directed energy deposition, including but not limited to laser engineering net shaping, directed light fabrication, direct metal deposition, and 3D laser cladding. For example, stereolithography can be used to directly fabricate one or more of the appliances herein. In some embodiments, stereolithography involves selective polymerization of a photosensitive resin (e.g., a photopolymer) according to a desired cross-sectional shape using light (e.g., ultraviolet light). The object geometry can be built up in a layer-by-layer fashion by sequentially polymerizing a plurality of object cross-sections. As another example, the appliance fabrication system(s) 910 may be configured to directly fabricate appliances using selective laser sintering. In some embodiments, selective laser sintering involves using a laser beam to selectively melt and fuse a layer of powdered material according to a desired cross-sectional shape in order to build up the object geometry. As yet another example, the appliance fabrication system(s) 910 may be configured to directly fabricate appliances by fused deposition modeling. In some embodiments, fused deposition modeling involves melting and selectively depositing a thin filament of thermoplastic polymer in a layer-by-layer manner in order to form an object. In yet another example, the appliance fabrication system(s) 910 may be configured to implement material jetting to directly fabricate appliances. In some embodiments, material jetting involves jetting or extruding one or more materials onto a build surface in order to form successive layers of the object geometry.

In some embodiments, the appliance fabrication system(s) 910 may include a combination of direct and indirect fabrication systems. In some embodiments, the appliance fabrication system(s) 910 may be configured to build up object geometry in a layer-by-layer fashion, with successive layers being formed in discrete build steps. Alternatively or in combination, the appliance fabrication system(s) 910 may be configured to use a continuous build-up of an object's geometry, referred to herein as “continuous direct fabrication.” Various types of continuous direct fabrication systems can be used. As an example, in some embodiments, the appliance fabrication system(s) 910 may use “continuous liquid interphase printing,” in which an object is continuously built up from a reservoir of photopolymerizable resin by forming a gradient of partially cured resin between the building surface of the object and a polymerization-inhibited “dead zone.” In some embodiments, a semi-permeable membrane is used to control transport of a photopolymerization inhibitor (e.g., oxygen) into the dead zone in order to form the polymerization gradient. Examples of continuous liquid interphase printing systems are described in U.S. Patent Publication Nos. 2015/0097315, 2015/0097316, and 2015/0102532, (corresponding to U.S. Patent Nos. corresponding to U.S. Pat. No. 9,205,601, 9,216,546, and 9,211,678) the disclosures of each of which are incorporated herein by reference in their entirety. As another example, the appliance fabrication system(s) 910 may be configured to achieve continuous build-up of an object geometry by continuous movement of the build platform (e.g., along the vertical or Z-direction) during the irradiation phase, such that the hardening depth of the irradiated photopolymer is controlled by the movement speed. Accordingly, continuous polymerization of material on the build surface can be achieved. Example systems are described in U.S. Pat. No. 7,892,474, the disclosure of which is incorporated herein by reference in its entirety.

In another example, the appliance fabrication system(s) 910 may be configured to extrude a composite material composed of a curable liquid material surrounding a solid strand. The composite material can be extruded along a continuous 3D path in order to form the object. Examples systems are described in U.S. Patent Publication No. 2014/0061974, corresponding to U.S. Pat. No. 9,511,543, the disclosures of which are incorporated herein by reference in its entirety.

In yet another example, the appliance fabrication system(s) 910 may implement a “heliolithography” approach in which a liquid photopolymer is cured with focused radiation while the build platform is continuously rotated and raised. Accordingly, the object geometry can be continuously built up along a spiral build path. Examples of such systems are described in U.S. Patent Publication No. 2014/0265034, corresponding to U.S. Pat. No. 9,321,215, the disclosures of which are incorporated herein by reference in its entirety.

The appliance fabrication system(s) 910 may include one or more elements of the aligner fabrication engine(s) 280 shown in FIG. 10.

The systems of the device planning environment 900 may operate to provide customized GUIs related to treatment planning. In some implementations, the treatment planning interface system(s) 904, the CPM system(s) 906 and the treatment planning system(s) 908 may operate to create treatment templates expressed according to treatment domain-specific protocols as follows. The CPM system(s) 906 may gather unstructured representations of treatment preferences from the treatment planning interface system(s) 904 through telephonic interviews, email exchanges, messages, conversations memorialized in notes, etc. A technician or an automated agent may use the tools on the CPM system(s) 906 to create treatment templates for a user in accordance with treatment domain-specific protocols. The CPM system(s) 906 may also validate the treatment templates to verify that the treatment templates accord with a given user and/or treatment outcome. The CPM system(s) 2806 may provide the treatment templates to the treatment planning system(s) 908 for storage and/or use in execution.

Additionally, the treatment planning interface system(s) 904, the treatment planning system(s) 908, and/or the appliance fabrication system(s) 910 may operate to provide treatment plans and/or appliances for a given patient. As noted herein, the treatment planning interface system(s) 104 may gather patient data. With the patient data, a user whose treatment preferences were previously memorialized with a treatment template may gather one or more treatment plans using the engines in the treatment planning system(s) 108. The treatment planning system(s) 108 may gather treatment templates and parse these treatment templates using the treatment domain-specific protocols in order to efficiently and effectively generate customized GUI elements that express treatment preferences in the context of a treatment plan. The user may interact with the treatment plan using the treatment planning interface system(s) 104. In various implementations, the user and/or the treatment planning system(s) 108 provide instructions to fabricate appliances with the appliance fabrication system 910.

FIG. 10 is a diagram showing an example of a system 1000; the system 1000 may be incorporated into a portion of another system (e.g., a general treatment planning system) and may therefore also be referred to as a sub-system. In any of the method and apparatuses described herein the system/sub-system may be invoked by a user control, such as a tab, button, etc., as part of treatment planning system, or may be separately invoked.

In FIG. 10, the system 1000 may include a plurality of engines and datastores. A computer system can be implemented as an engine, as part of an engine or through multiple engines. As used herein, an engine may include one or more processors or a portion thereof. A portion of one or more processors can include some portion of hardware less than all of the hardware comprising any given one or more processors, such as a subset of registers, the portion of the processor dedicated to one or more threads of a multi-threaded processor, a time slice during which the processor is wholly or partially dedicated to carrying out part of the engine's functionality, or the like. As such, a first engine and a second engine can have one or more dedicated processors or a first engine and a second engine can share one or more processors with one another or other engines. Depending upon implementation-specific or other considerations, an engine can be centralized or its functionality distributed. An engine can include hardware, firmware, or software embodied in a computer-readable medium for execution by the processor. The processor transforms data into new data using implemented data structures and methods, such as is described with reference to the figures herein.

The engines described herein, or the engines through which the systems and devices described herein can be implemented, can be cloud-based engines. As used herein, a cloud-based engine is an engine that can run applications and/or functionalities using a cloud-based computing system. All or portions of the applications and/or functionalities can be distributed across multiple computing devices, and need not be restricted to only one computing device. In some embodiments, the cloud-based engines can execute functionalities and/or modules that end users' access through a web browser or container application without having the functionalities and/or modules installed locally on the end-users' computing devices.

As used herein, datastores are intended to include repositories having any applicable organization of data, including tables, comma-separated values (CSV) files, traditional databases (e.g., SQL), or other applicable known or convenient organizational formats. Datastores can be implemented, for example, as software embodied in a physical computer-readable medium on a specific-purpose machine, in firmware, in hardware, in a combination thereof, or in an applicable known or convenient device or system. Datastore-associated components, such as database interfaces, can be considered “part of” a datastore, part of some other system component, or a combination thereof, though the physical location and other characteristics of datastore-associated components is not critical for an understanding of the techniques described herein.

Datastores can include data structures. As used herein, a data structure is associated with a particular way of storing and organizing data in a computer so that it can be used efficiently within a given context. Data structures are generally based on the ability of a computer to fetch and store data at any place in its memory, specified by an address, a bit string that can be itself stored in memory and manipulated by the program. Thus, some data structures are based on computing the addresses of data items with arithmetic operations; while other data structures are based on storing addresses of data items within the structure itself. Many data structures use both principles, sometimes combined in non-trivial ways. The implementation of a data structure usually entails writing a set of procedures that create and manipulate instances of that structure. The datastores, described herein, can be cloud-based datastores. A cloud-based datastore is a datastore that is compatible with cloud-based computing systems and engines.

The system/sub-system 1000 may include or be part of a computer-readable medium, and may include a user interface (I/F) engine 1020. The user I/F engine 1020 may allow a user to interact with one or more software modules. As used herein, a user may refer to a doctor, dentist, or other clinician associated with determining, providing or generating a treatment plan. In some examples, the user I/F engine 1020 may display interactive dialog boxes to the user. In turn, the user may interact with the dialog boxes to indicate choices and/or decisions regarding a patient's treatment plan and/or a doctor's treatment preferences. In some variations, the dialog boxes may enable the user to modify part or portions of the patient's treatment plan and/or the doctor's treatment preferences. In some other variations, the user I/F engine 1020 may enable the user to modify the patient's treatment plan and/or the doctor/s treatment preferences within a data structure (e.g., a datastore). Thus, the user I/F engine 1020 may enable the user to read, modify, and/or write data within any feasible datastore. Alternatively, or in addition, the user I/F engine 1020 may enable the user to review, accept, reject, and/or apply any modifications to treatment preferences or treatment plans.

The system/subsystem 1000 may also include a treatment plan generator engine 1040. The treatment plan generator engine 1040 may generate a treatment plan to provide dental or orthodontic treatment for a patient. In some examples, the treatment plan generator engine 1040 may generate a patient's treatment plan based at least in part on patient data and a doctor's treatment preferences. Thus, the treatment plan generator engine 1040 may accept or receive a data structure including treatment preferences and a digital model of the patient's teeth to generate a treatment plan. In some variations, the treatment plan generator engine 1040 may store modified treatment parameters. The modified treatment parameters may be based on a doctor's treatment preferences as modified by the doctor (or any other feasible user) via the user I/F engine 1020. In some examples, the treatment plan generator engine 1040 may store the treatment plan in an applicable datastore. Furthermore, the modified treatment parameters may be stored in a dental protocol language.

The system/subsystem 1000 may also include a display treatment plan engine 1070. The display treatment plan engine 1070 may enable the doctor to review and/or approve a patient's treatment plan, particularly a treatment plan as modified via the user I/F engine 1020. In some examples, the display treatment plan engine 1070 may display before, during, and after representations or visualizations of a patient's teeth as treated by one or more aligners that are based on a treatment plan generated by the treatment plan generator engine 1040.

The system/subsystem 1000 may also include an aligner fabrication engine 1080. The aligner fabrication engine 1080 may process patient data 1030 and a treatment plan to generate one or more (in some cases a series of) aligners, including clear dental aligners to treat a patient's teeth. The aligners may be generated by any feasible method. In some variations, the aligner fabrication engine 1080 may generate images or renderings associated with one or more dental aligners. These images/renderings may be displayed to the user through the display treatment plan engine 1070. In some examples, the aligners may be fabricated by the appliance fabrication system 910 of FIG. 9.

The system/subsystem 1000 may include any number of datastores. For example, the system/subsystem 1000 may include a data structure of treatment parameters 1010. In some variations, the data structure may be expressed in a dental protocol language. The use of a dental protocol language (sometimes referred to as a domain-specific orthodontic treatment language) that is both human and machine readable, and is tailored to orthodontic treatment provides a high level of flexibility and efficiency in orthodontic treatment planning and orthodontic device fabrications. For example, the dental protocol language enables automation of many different orthodontic treatment planning protocols, and facilitates the communication between users (e.g., doctors), technicians and R&D personnel. It adds more flexibility than simple parameter files because it includes semantics for conditional statement, and because it exposes more configuration options. The dental protocol language may be used for editing and for visualizing the treatment planning protocol (TPP), and may therefore be concise and easy to understand. The dental protocol language scripts may be automatically translated into executable code in an interpreted language.

The treatment parameters may describe a doctor's treatment preferences. In some examples, the treatment parameters may be predefined. For example, the doctor's treatment preferences may be based on a predefined personalized plan that may have been customized by and/or for the doctor to indicate one or more treatment preferences. In some examples, the doctor's treatment preferences may be predefined based on prior treatment plans approved by the doctor as used on other patients. In some other examples, the doctor's treatment preferences may be predefined based on those of a Key Opinion Leader (KOL). A Key Opinion Leader's treatment preferences may be those associated with a particular clinician. In some other examples, the doctor's treatment preferences may be predefined based on Regional Automated Defaults (RAD). RAD preferences may be associated with a geographic or other region with which the doctor wishes to follow. In still other examples, the doctor's treatment preferences may be predefined based on Dental Service Organization (DSO) templates. A DSO template may provide treatment preferences that are associated with and suggested by a doctor's DSO. In some examples, the predefined treatment parameters may be stored or maintained in a library of clinician treatment preferences. The treatment parameters may be stored, recorded, and/or indexed by the clinician.

The system/subsystem 1000 may include a datastore of patient data 1030. The patient data 1030 may include any feasible patient data including scans, x-rays, dental imaging, patient physiological details (age, weight, gender), and the like. In some examples, patient data may include a digital model of the patient's teeth.

The system/subsystem 1000 may include a datastore of modified treatment parameters 1050. For example, the treatment plan generator engine 1040 may store modified treatment parameters in the modified treatment parameter datastore 1050. In some cases, the modified treatment parameters may be appended into the modified treatment parameter datastore 1050. The treatment parameters may be modified through the user I/F engine 1020 interacting the data structure of treatment parameters 1010.

The system/subsystem 1000 may include a treatment plan datastore 1060. The treatment plan datastore 1060 may include a treatment plan for a patient based on treatment parameters in the modified treatment parameter datastore 1050.

FIG. 11 shows an example user interface 1100 displaying selection choices for modifying treatment parameters. As described herein, a doctor's treatment parameters may be associated with one or more pre-defined treatment preferences. By interacting with the user interface 300, a doctor or other user may easily modify any of the pre-defined treatment preferences. The user interface 1100 may include any feasible number of interaction regions with which the doctor may interact to modify treatment preferences.

As shown, the user interface 1100 may include four interaction regions 1102-1105. In other examples, the user interface 1100 may include fewer than, or more than four interaction regions 1102-1105. Each interaction region may be associated with modifying or changing one or more aspects of the doctor's treatment preferences.

A first interaction region 1102 may enable a doctor to specify or modify treatment parameters associated with treating dental arches. For example, the first interaction region 1102 may allow the doctor to specify or modify treatment parameters for performing treatment on an upper arch, a lower arch, or both arches. A second interaction region 1103 may enable a doctor to specify or modify treatment parameters associated with one or more particular teeth. For example, through the second interaction region 1103 may enable the doctor to specify one or more tooth extractions and/or one or more tooth restrictions. A tooth restriction may enable the doctor to specify tooth movement restrictions and/or tooth attachment restrictions. A third interaction region 1104 may enable a doctor to specify eruption compensation characteristics for one or more teeth. A fourth interaction region 1105 may enable the doctor to specify or modify treatment parameters associated with following another protocol. For example, different doctor protocols may be displayed and may be selected. In response, a particular doctor protocol may be used to modify treatment parameters for a patient's treatment plan.

Thus, through interactions with one or more interaction regions, the user can modify one or more treatment parameters. In many cases, the treatment parameters may be written, expressed, and/or saved in a dental protocol language. Therefore, through the interaction regions, a user can modify treatment parameters without knowledge of the dental protocol language.

### Domain-Specific Orthodontic Treatment Language

A domain-specific orthodontic treatment language (sometimes referred to as a dental protocol language) may include syntax and grammar that is specific to orthodontic treatments. For example, a domain-specific orthodontic treatment language may include grammar specific to clinical settings, which may affect one or more of the orthodontic treatment planning phases. The domain-specific orthodontic treatment language may include a verb and a noun, and optional arguments. For example: “disable class_correction”; “restrict movements (teeth: molars)”; “limit ipr (teeth: anteriors, max_amount: 0.30 mm)”; “set filters (any_product, open_bite, overjet, ipr, attachments)”; “put hook(on: upper canines)”.

The domain-specific orthodontic treatment language may also include conditional statements, including conditional “if” statements that refer to the initial position, final position, treatment goals or existence of teeth or other conditions in the dental arch and treatment of the dental arch. For example: “if (initially open_bite>0.5 mm) . . . ”; “if (performing intrusion(upper anteriors)>0.5 mm) . . . ”; “if (performing extrusion(lower molars)>0.5 mm and initially posterior_open_bite>0.3 mm) . . . ”.

The domain-specific orthodontic treatment language may also include values that are given in units appropriate to the orthodontic treatment such as: millimeters, degrees or percentages, etc.: “50%”; “1.5 mm”; “−0.5mm”; “30 degrees”.

The domain-specific orthodontic treatment language may reference directly tooth types and number, and may use ranges of teeth and individual tooth names, such as: “canines”; “molars”; “canines and molars”; “upper left molars and lower left molars”; “upper 2nd premolar and lower 2nd premolar”; “primary second molars”; “upper primary centrals”.

The domain-specific orthodontic treatment language may include loops, such as “for” loops, to repeat a set of instructions over a range of teeth, quadrants or for each jaw. For example:

```
  for each tooth (of: canines) {
    if (performing rotation > 30 degrees) // Repeated for each single tooth
independently
    postpone movement(direction: rotation);
      put attachment(teeth: mesial, type: optimized);
    }
```

And:

```
  for each quadrant (of: [upper left, upper right]) { ... }
    for each jaw {
      if (performing intrusion(teeth: anteriors) > 0.5mm) {
      put attachment(on: canine, type: CRT, size: 3mm, direction: horizontal,
        min_distance_from_occlusal: 1mm);
      put attachment(on: 1st premolar, type: CRT, size: 3mm, direction:
horizontal, min_distance_from_occlusal: 1mm);
      }
    }
```

The domain-specific orthodontic treatment language may include lists (e.g., sequences of entities where the order matters), such as: “apply movement_separation(teeth: anteriors, order: [lingual_root_torque, intrusion])”; “apply sequential_movement(movement: mesialization, overlap: 0%, order: [incisors, canines])”.

The domain-specific orthodontic treatment language may also include nested code blocks, such as:

```
  if (performing extrusion(upper molars) > 0.5mm) {
    if (not performing distalization(upper molars)) {
    apply sequential_movement(direction: extrusion, teeth: [upper
premolars, upper molars]);
    } else {
    apply sequential_movement(direction: extrusion, teeth: [upper
molars, upper premolars]);
    }
  }
```

The domain-specific orthodontic treatment language may also reuse different templates or parts of templates, and may call them by name. For example: “use template Dr. XYZ”; “use template XYZ”.

The domain-specific orthodontic treatment language may include comments that are not parsed by the orthodontic treatment planning algorithms, and are only used for communication with the user or other stakeholders.

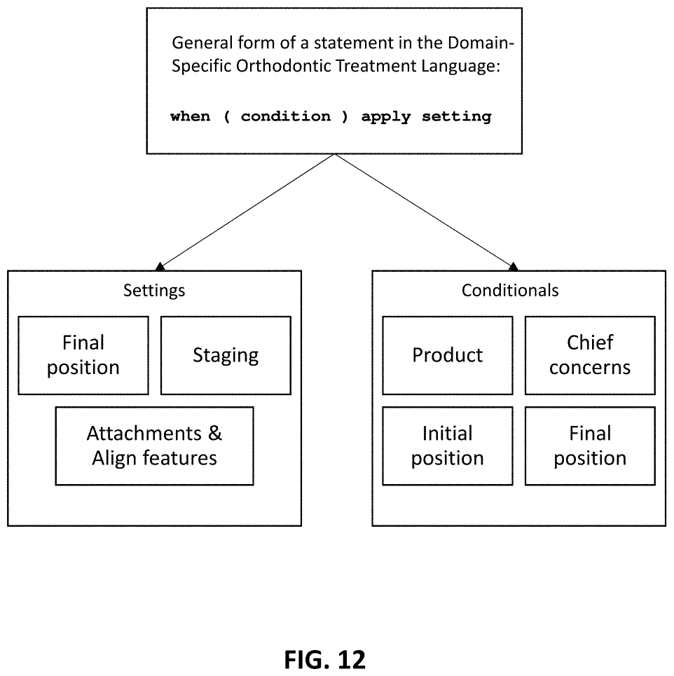

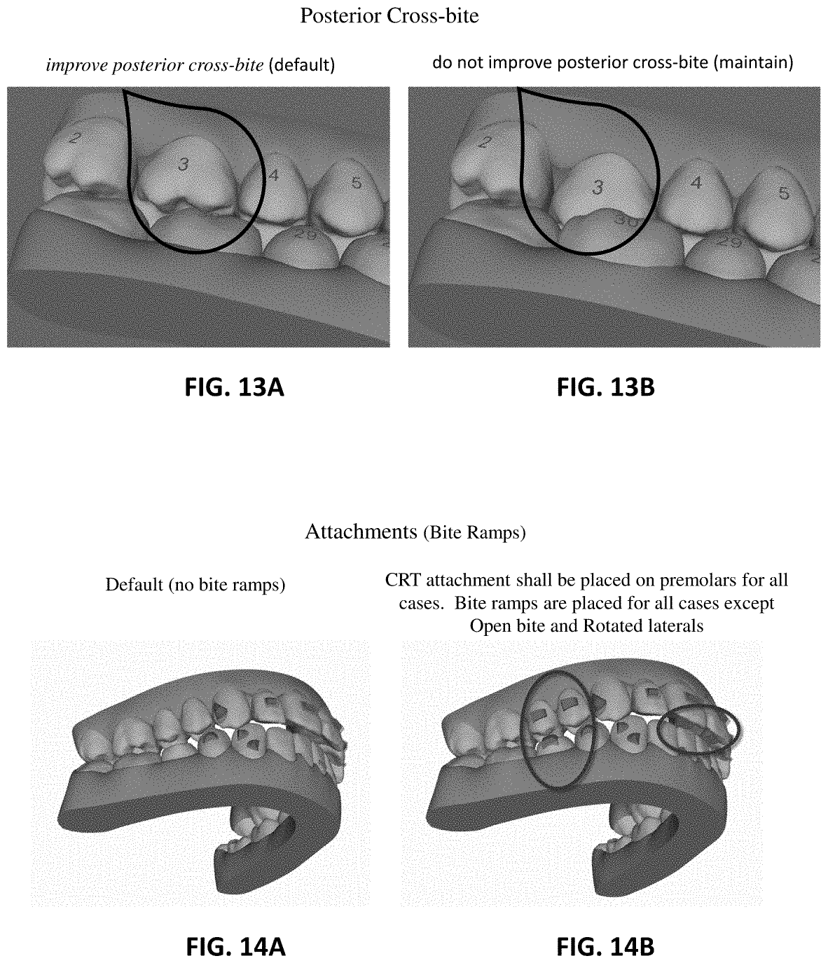

For example, FIG. 12 shows an example of overview of a general statement of a domain-specific orthodontic treatment language, showing various settings (e.g., positions) and conditionals. The domain-specific orthodontic treatment language is particularly well suited and specifically configured to include and encode user-specific preferences for orthodontic treatments. FIGS. 13A-13B, 14A-14B, 15A-15B, 16A-16B and 17A-17B illustrate examples of some parameters that may be considered user preferences and may be directly encoded and referenced in the domain-specific orthodontic treatment language.

For example, FIGS. 13A-13B show an example of a posterior cross-bite preference that may be set by the domain-specific orthodontic treatment language. FIG. 13A shows the default preference, in which the orthodontic treatment planning engine(s) will automatically try and improve the posterior cross-bite, as shown. Alternatively, the user may select to not improve the posterior cross-bite, as shown in FIG. 13B, but instead may maintain it.

In FIGS. 14A-14B the domain-specific orthodontic treatment language may allow the user to indicate a preference for include bite ramp attachments on the premolars or not. In FIG. 14A, the default is not to include premolar bite-ramp attachments; while in FIG. 14B premolar bite ramp attachments are included. In some variations, as in any of the preferences, the user may select one or more conditions under which the orthodontic feature (e.g., premolar bite ramps in this example) are included or not included. For example, the user may specify that premolar bite ramps are to be included in all cases except where there is an open bite and rotated laterals.

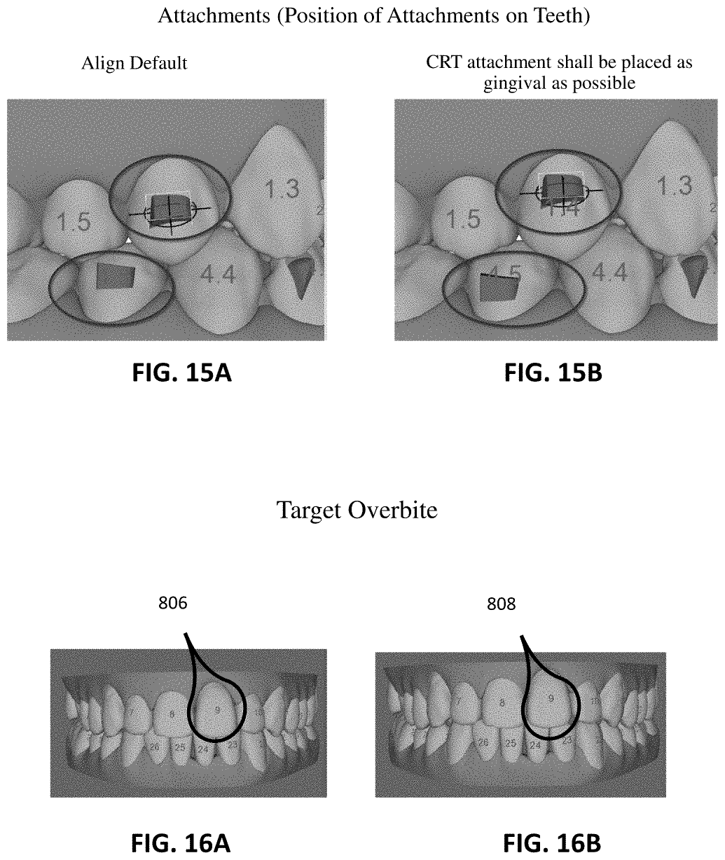

FIGS. 15A-15B illustrate the preference options for the domain-specific orthodontic treatment language regarding the position of attachments on the patient's teeth. In this example, the default preference may be to center the attachments (which may anchor or couple with sites on the dental appliance to help retain the appliance on the teeth), as shown in FIG. 15A. The user interface may show rotation (degrees x, y, z), crown translation (x, y, z mm), root translation (e.g., x, y, z mm), root apex movement (x, y, mm), FACC measurements (medial to distal width, buccal to lingual width, mm), and/or attachment (type, description, visibility), etc. In some variations the user may indicate in the domain-specific orthodontic treatment language that the attachments should be placed as close to the gingiva as possible, as shown in FIG. 15B.

FIGS. 16A-16B illustrate examples of preferences for target overbite correction in the domain-specific orthodontic treatment language. For example, in FIG. 16A, the default 806 parameter is that the target overbite may be between, e.g., 1.3 and 1.6 mm (in FIG. 16A, the final position is 1.45529 mm), e.g., about 1.5 mm. The user may select an alternative range. For example, the user may select in the domain-specific orthodontic treatment language a target overbite of a given value in mm (e.g., about 0.1 mm, 0.2 mm, 0.3 mm, 0.4 mm, 0.5 mm, 0.6 mm, 0.7 mm, 0.8 mm, etc.). The user interface may display sate (initial, auto-setup and final, in mm). In FIG. 16B, the target ideal overbite 808 of a given value may be in mm (e.g., 0.1 mm, 0.2 mm, 0.3 mm, 0.4 mm, 0.5 mm, 0.6 mm, 0.7 mm, 0.8 mm, etc.)

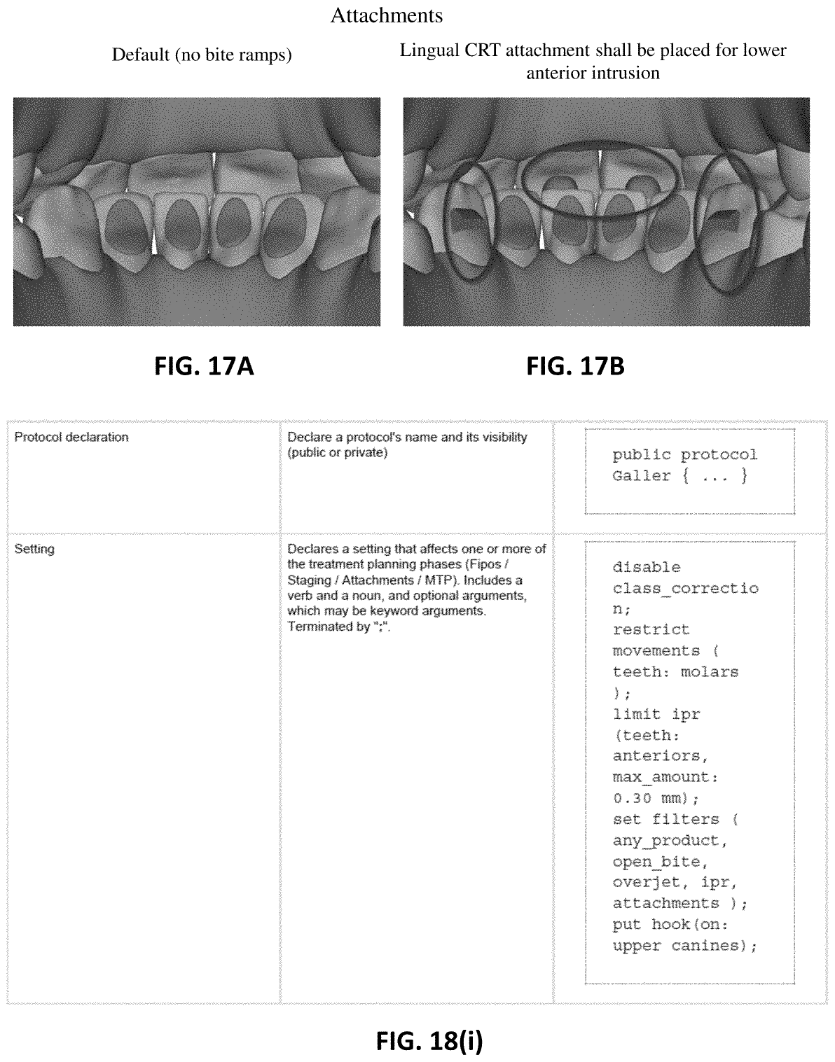

FIGS. 17A-17B illustrate the selection of user preference of lingual bite ramp attachments in the domain-specific orthodontic treatment language. In FIG. 17A, the default preference is shown, without lingual bite ramps for lower anterior intrusion. In contrast, as shown in FIG. 17A, the user may indicate in the domain-specific orthodontic treatment language that the orthodontic treatment plan should include lingual bite ramps for lower anterior intrusion.

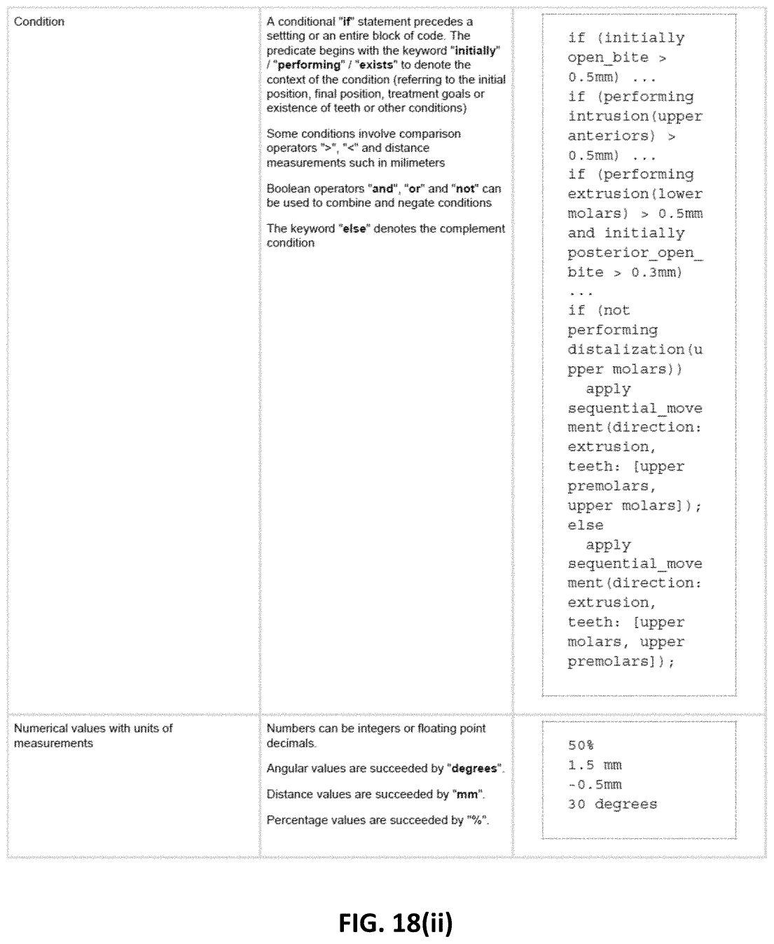

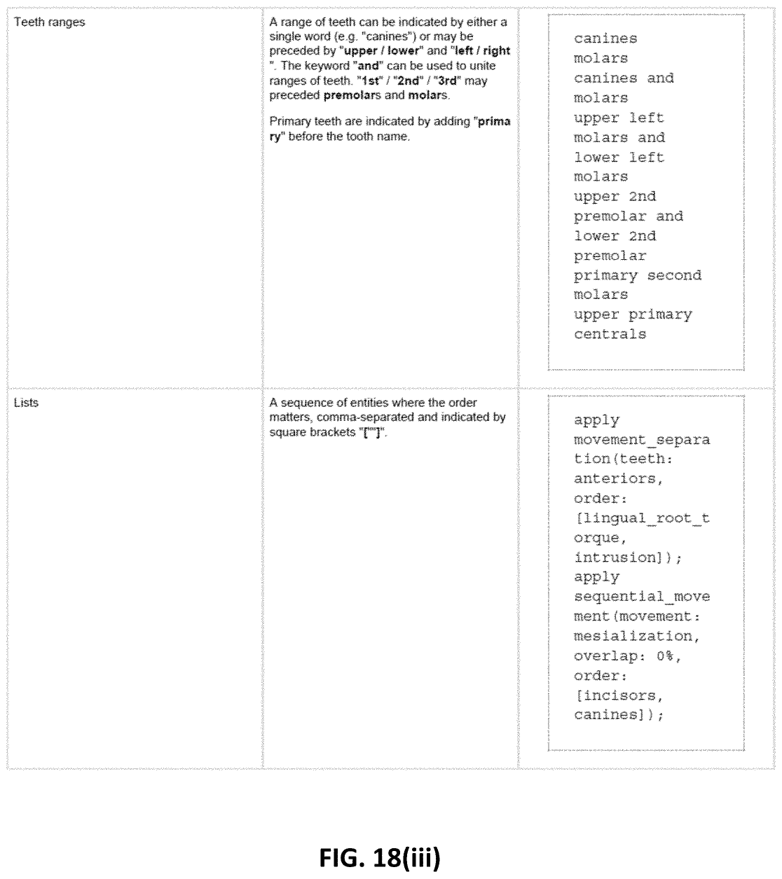

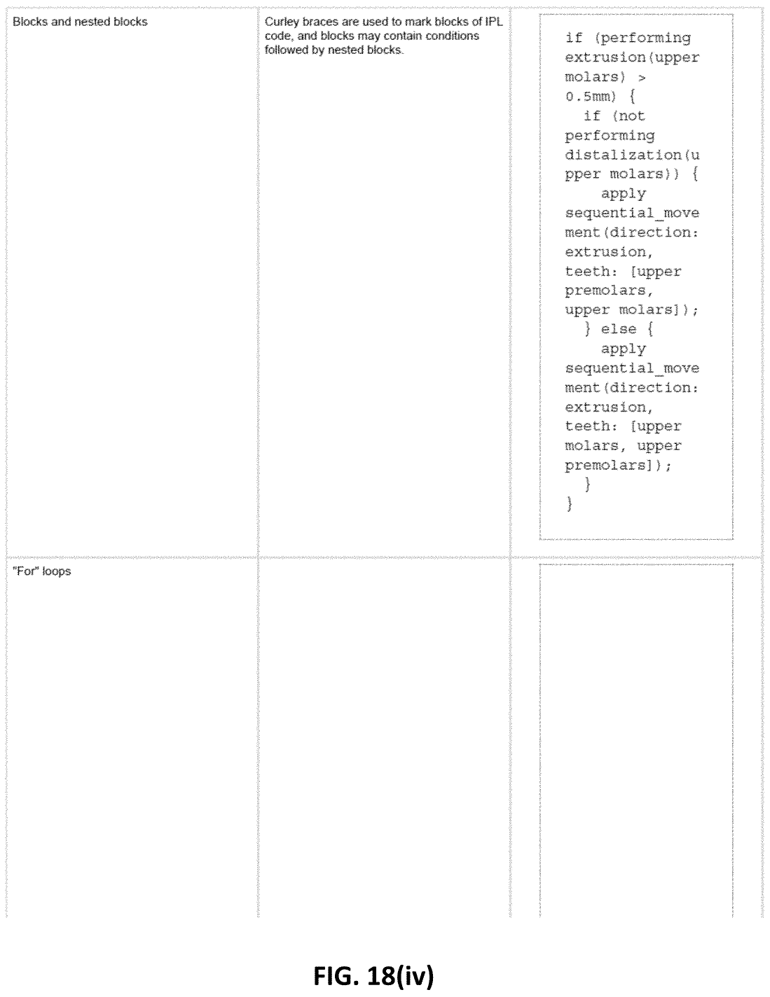

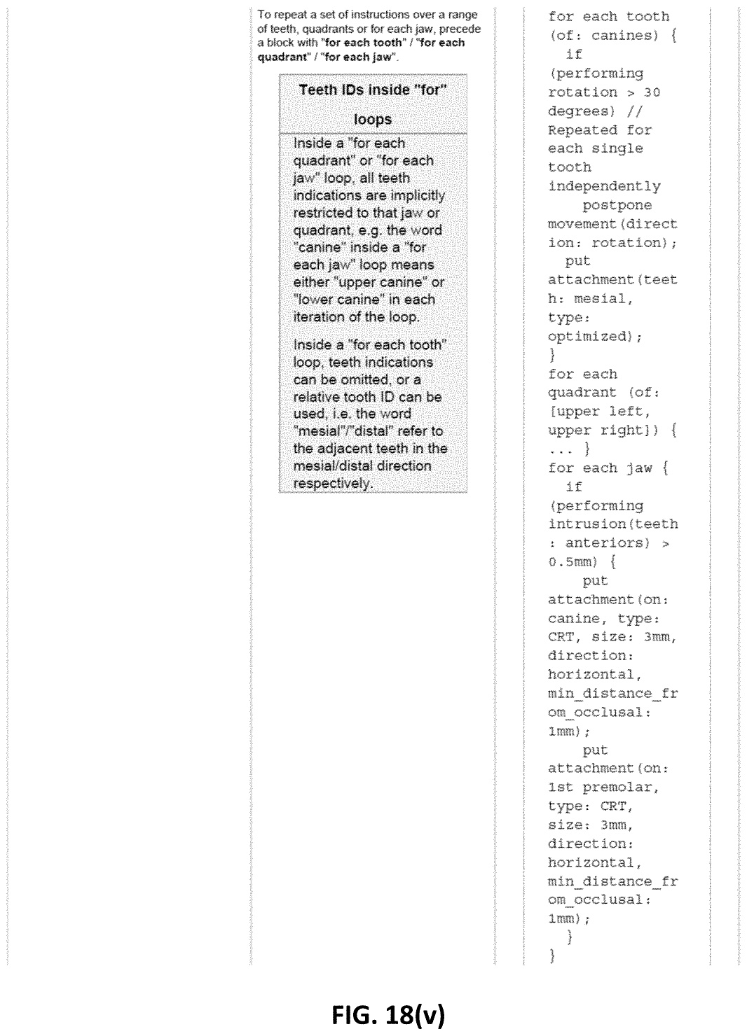

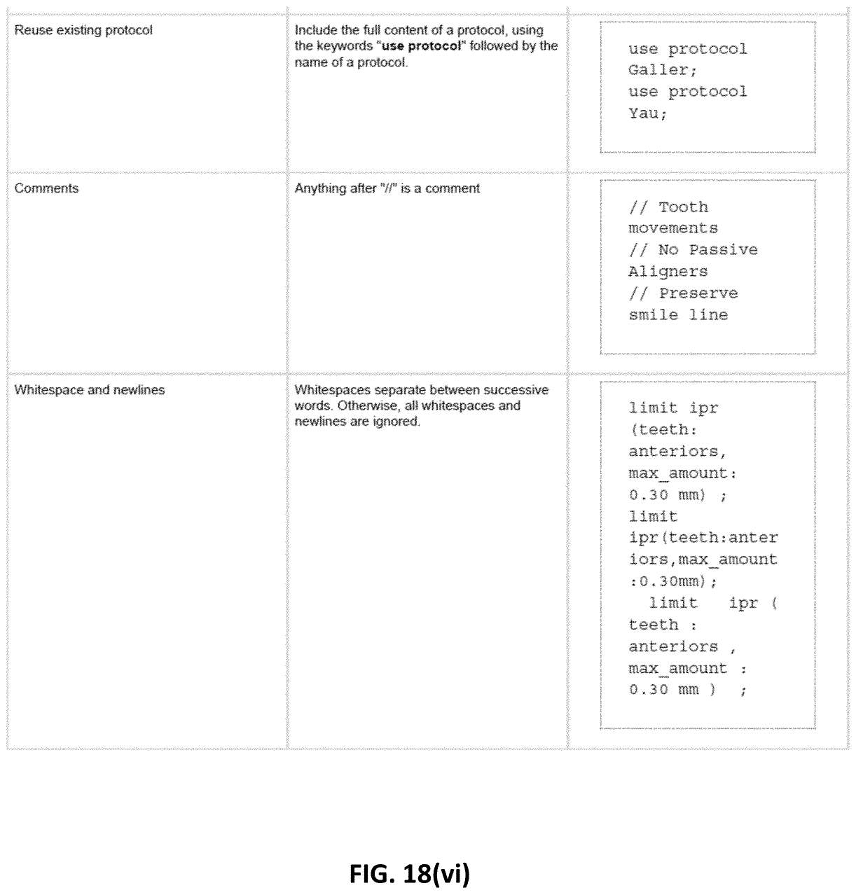

Multiple other preferences and conditions may be indicated by the domain-specific orthodontic treatment language. The table shown in FIGS. 18(i)-18(vi) illustrates examples of the grammar and syntax for a domain-specific orthodontic treatment language. This table illustrates conditional (e.g., “if”) language using the orthodontic treatment specific context, as well as numerical ranges, teeth ranges, and list.

FIG. 19 shows an example of a method 1900 for modifying a treatment plan. Some examples may perform the operations described herein with additional operations, fewer operations, operations in a different order, operations in parallel, and some operations differently. The method 1900 is described below with respect to the system 1000 of FIG. 10, however, the method 1900 may be performed by any other suitable system or device.

The method 1900 begins in block 1902 as a doctor's treatment preferences are retrieved. For example, the treatment plan generator engine 1040 may access the doctor's treatment preferences that may be stored in a datastore as a data structure of treatment parameters 1010. The doctor's treatment preferences may be based on a library of clinician treatment preferences. The library of clinician treatment preferences may include treatment preferences associated with KOLs, RADs, DSOs, or any other feasible group of treatment preferences. In some examples, the treatment preferences may be associated with prior treatment plans used and/or approved by the doctor for other patients. One or more of the retrieved doctor's treatment preferences may be presented and/or displayed to the doctor.

Next, in block 1904 the system 1000 receives user input for treatment preference modifications. For example, the user may be presented a user interface with one or more choices to modify one or more treatment parameters 1010. Through the user interface, the user may select one or more treatment parameters to modify. For example, the user may select an interaction region on the user interface that may be associated with modifying a group or subset of treatment parameters.

Next, in block 1906, the system 1000 may modify the treatment parameters in accordance with user input. For example, user input (received in block 1904) may be used to modify the doctor's treatment parameters. In some examples, the treatment plan generator engine 1040 may modify treatment parameters and store the modified treatment parameters in a data structure. In some variations, the modified treatment parameters may be stored in a high layer of a data structure, such as in a data structure in the modified treatment parameter datastore 1050.

Next, in block 1910, the system 1000 may generate a treatment plan. The generated treatment plan may be based on the modified treatment parameters and, in some cases, the patient data. In some examples, the patient data may be the patient data in the patient data datastore 1030. Next, in optional block 1911, the treatment plan may be displayed on a user interface. Through the user interface, a doctor can approve, reject, or modify the treatment plan and/or related treatment parameters. In some variations, the method 1900 may optionally return to block 1906. This return enables an iteration or repeating of one or more actions or steps so that the user can refine a treatment plan.

Next, in block 1912 the system 1000 may fabricate aligners. For example, the aligner fabrication engine 1080 may fabricate a patient's dental aligners based on patient data 1030 and the data in the treatment plan datastore 1060. (Note that the treatment plan may be based on modified treatment parameters, such as the modified treatment parameters in the modified treatment parameter datastore 1050.) In some variations, the aligners may be displayed on a user interface allowing a doctor to approve, reject, or modify the treatment plan and/or related treatment parameters.

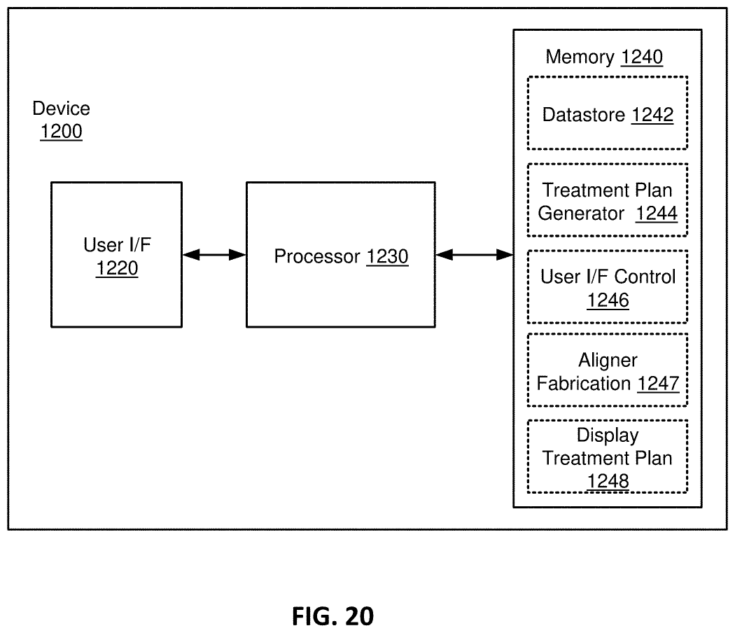

FIG. 20 shows a block diagram of a device 1200 that may be an example of the system 1000 of FIG. 10. The device 1200 may include a user interface 1220, a processor 1230, and a memory 1240.

The user interface 1220, which is coupled to the processor 1230, may be used to interface with any feasible user device. For example, the user interface 1220 may be coupled to and interface with a display (not shown). In some examples, the display may be used to show treatment plans, treatment parameters, aligners, predicted tooth movements, and the like. The user interface 1220 may be coupled to a device to receive user input. For example, the user interface 1220 may be coupled to a touch screen, a keyboard, mouse, or any other feasible device that may receive user input. In some examples, the user interface 1220 may include one or more wireless transceivers to send and receive wireless data associated with any feasible user interface. Thus, the user interface 1220 may include any feasible Wi-Fi, Bluetooth, cellular transceiver. In this manner, a wireless connection may be provided to a remote display, keyboard, touch screen, or the like.

The processor 1230, which is also coupled to the memory 1240, may be any one or more suitable processors capable of executing scripts or instructions of one or more software programs stored in the device 1200 (such as within memory 1240).

The memory 1240 may include a datastore 1242 that may be used to locally store any feasible data. For example, the datastore 1242 may include treatment patient data, a data structure of treatment parameters, one or more modified treatment parameters, and a patient's treatment plan, or any other feasible treatment information.

The memory 1240 may also include a non-transitory computer-readable storage medium (e.g., one or more nonvolatile memory elements, such as EPROM, EEPROM, Flash memory, a hard drive, etc.) that may store the following software modules: a treatment plan generator module 1244 to generate and store a patient's treatment plan; a user interface (I/F) control module 1246 to control the user interface 1220; an aligner fabrication module 1247 to generate data that, in turn, may be used to manufacture and/or create dental aligners; and a display treatment plan module 1248 to display a treatment plan.

Each software module includes program instructions that, when executed by the processor 1230, may cause the device 1200 to perform the corresponding function(s). Thus, the non-transitory computer-readable storage medium of memory 1240 may include instructions for performing all or a portion of the operations described herein.

The processor 1230 may execute the treatment plan generator module 1244 to generate and store a patient's treatment plan. In some examples, execution of the treatment plan generator module 1244 may also modify treatment preferences, generate modified treatment parameters, and/or generate a treatment plan based on modified treatment preferences. Execution of the treatment plan generator module 1244 may access any applicable data (e.g., treatment preferences, modified treatment parameters, client data, and the like) in the datastore 1242.

The processor 1230 may execute the user I/F control module 1246 send or receive user data through the user interface 1220. For example, execution of the user I/F control module 1246 may cause treatment plan information and/or aligner information to be displayed to the user. In another example, execution of the user I/F control module 1246 may cause the user interface 1220 to receive user input. Example user input may indicate the approval, rejection, or modification of patient data, treatment plans, and/or treatment preferences.

The processor 1230 may execute the aligner fabrication module 1247 to generate aligner data that, in turn, may be used to fabricate one or more aligners. For example, execution of the aligner fabrication module 1247 may use patient data and treatment preferences stored in the datastore 1242 to generate aligner data for a series of aligners. In some examples, aligner data may be displayed to the user via the user I/F control module 1246 and the user interface 1220.

The processor 1230 may execute the display treatment plan module 1248 to display a treatment plan to the user. For example, execution of the display treatment plan module 1248 may cause a treatment plan to be displayed via execution of the user I/F control module 1246 and the user interface 1220. In some variations, execution of the display treatment plan module 1248 may display dental aligners, predicted tooth movements, treatment preferences, treatment parameters, modified treatment parameters, interactive regions, or the like.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like. For example, any of the methods described herein may be performed, at least in part, by an apparatus including one or more processors having a memory storing a non-transitory computer-readable storage medium storing a set of instructions for the processes(s) of the method.

While various embodiments have been described and/or illustrated herein in the context of fully functional computing systems, one or more of these example embodiments may be distributed as a program product in a variety of forms, regardless of the particular type of computer-readable media used to actually carry out the distribution. The embodiments disclosed herein may also be implemented using software modules that perform certain tasks. These software modules may include script, batch, or other executable files that may be stored on a computer-readable storage medium or in a computing system. In some embodiments, these software modules may configure a computing system to perform one or more of the example embodiments disclosed herein.

As described herein, the computing devices and systems described and/or illustrated herein broadly represent any type or form of computing device or system capable of executing computer-readable instructions, such as those contained within the modules described herein. In their most basic configuration, these computing device(s) may each comprise at least one memory device and at least one physical processor.

The term “memory” or “memory device,” as used herein, generally represents any type or form of volatile or non-volatile storage device or medium capable of storing data and/or computer-readable instructions. In one example, a memory device may store, load, and/or maintain one or more of the modules described herein. Examples of memory devices comprise, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

In addition, the term “processor” or “physical processor,” as used herein, generally refers to any type or form of hardware-implemented processing unit capable of interpreting and/or executing computer-readable instructions. In one example, a physical processor may access and/or modify one or more modules stored in the above-described memory device. Examples of physical processors comprise, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor.

Although illustrated as separate elements, the method steps described and/or illustrated herein may represent portions of a single application. In addition, in some embodiments one or more of these steps may represent or correspond to one or more software applications or programs that, when executed by a computing device, may cause the computing device to perform one or more tasks, such as the method step.

In addition, one or more of the devices described herein may transform data, physical devices, and/or representations of physical devices from one form to another. Additionally or alternatively, one or more of the modules recited herein may transform a processor, volatile memory, non-volatile memory, and/or any other portion of a physical computing device from one form of computing device to another form of computing device by executing on the computing device, storing data on the computing device, and/or otherwise interacting with the computing device.

The term “computer-readable medium,” as used herein, generally refers to any form of device, carrier, or medium capable of storing or carrying computer-readable instructions. Examples of computer-readable media comprise, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

The processor as described herein can be configured to perform one or more steps of any method disclosed herein. Alternatively or in combination, the processor can be configured to combine one or more steps of one or more methods as disclosed herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A method comprising:
presenting a dental practitioner with a graphical user interface including a menu of textual rules specific to orthodontic treatment planning, wherein the rules refer to a collection of instructions in a domain-specific treatment language;
receiving, from the dental practitioner, selections from the menu of textural rules to form a practitioner-specific subset of textural rules;
applying the practitioner-specific subset of textural rules to a test dental data set to generate a test treatment plan;
displaying the test treatment plan to the dental practitioner;
modifying, based on input from the dental practitioner, the practitioner-specific subset of textural rules; and
storing the practitioner-specific subset of textural rules to apply to one or more sets of patient data.

2. The method of claim 1, wherein the menu of textual rules comprises rules specific to tooth leveling, gap spacing, crowding, extraction, interproximal reduction, overjet, overbite, cross bite, attachments, and anterior-to-posterior correction.

3. The method of claim 1, further comprising reconciling any conflict between the rules of the practitioner-specific subset of textural rules.

4. The method of claim 1, wherein modifying comprises receiving modifications to the test treatment plan and modifying the practitioner-specific textural rules accordingly.

5. The method of claim 1, wherein modifying comprises receiving dental practitioner edits to the practitioner-specific subset of textural rules directly.

6. The method of claim 1, further comprising generating a treatment plan specific to a patient by applying the practitioner-specific subset of textural rules to the patient's dental data.

7. The method of claim 6, further comprising fabricating dental appliances based on the treatment plan.

8. The method of claim 1, wherein the graphical user interface comprises a user dialog box including a selectable list of the textual rules specific to orthodontic treatment planning.

9. The method of claim 1, wherein the menu of textual rules specific to orthodontic treatment planning comprises a curated list of textural rules organized by rank.

10. The method of claim 1, wherein the rules are taken from a curated rules database and selected based on one or more characteristic of the dental practitioner.

11. The method of claim 10, wherein the characteristic of the dental practitioner comprises a size of the dental practitioner's practice, a number of dental aligner cases performed by the dental practitioner, and a geographic location of the dental practitioner.

12. The method of claim 1, wherein applying the practitioner-specific subset of textural rules to the test dental data set to generate the test treatment plan comprises generating a plurality of dental aligners corresponding to each of a plurality of stages of the test treatment plan.

13. The method of claim 1, further comprising providing a patient data set from the dental practitioner as the test dental data set.

14. The method of claim 1, wherein the test dental data set comprises a standardized dental data set.

15. The method of claim 1, wherein displaying the test treatment plan to the dental practitioner comprises displaying the menu of textural rules in a first window and displaying an image of teeth corresponding to the test dental data set at one or more stages of the test treatment plan in a second window so that the dental practitioner may modify the selection of textural rules from the menu of textural rules in the first window and may modify a view of the image of teeth corresponding to the test dental data set at one or more stages of the test treatment plan in the second window.

16. A method comprising:
presenting a dental practitioner with a graphical user interface comprising a first window including a menu of textual rules specific to orthodontic treatment planning, wherein the rules refer to a collection of instructions in a domain-specific treatment language;
receiving, from the dental practitioner, selections from the menu of textural rules to form a practitioner-specific subset of textural rules;
reconciling any conflict between the rules of the practitioner-specific subset of textural rules;
applying the practitioner-specific subset of textural rules to a test dental data set to generate a test treatment plan;
displaying the test treatment plan to the dental practitioner in a second window, wherein the second window displays an image of teeth corresponding to the test dental data set at one or more stages of the test treatment plan;
modifying, based on input from the dental practitioner, the practitioner-specific subset of textural rules; and
generating a treatment plan specific to a patient by applying the practitioner-specific subset of textural rules to the patient's dental data.

17. A method of generating a treatment plan for an orthodontic treatment, the method comprising:
presenting a clinician with predefined treatment parameters for generating a treatment plan, wherein the predefined treatment parameters comprise a data structure in a dental protocol language;
accepting, through a user interface, one or more modifications to the predefined treatment parameters;
appending the one or more modifications to the data structure as a higher layer;
transferring the data structure and a digital model of a patient's teeth to a treatment plan generating module, wherein the treatment plan generating module modifies the treatment parameters based on the higher layer and generates a modified set of treatment parameters; and
displaying the treatment plan to the clinician for approval.

18. The method of claim 17, wherein the modifications are encoded in the dental protocol language.

19. The method of claim 17, further comprising repeating the steps of presenting the clinician with the predefined treatment parameters and accepting one or more modifications following the display of the treatment plan to the clinician.

20. The method of claim 17, further comprising forming a series of aligners based on the approved treatment plan.

21. The method of claim 17, wherein presenting the clinician with the predefined treatment preferences comprises generating or receiving the predefined treatment preferences from a library of clinician treatment preferences indexed by the clinician.

22. The method of claim 17, wherein the predefined treatment preferences are based on prior treatment plans approved by the clinician for other patients.
